(19) United States
(12) Patent Application Publication
(10) Pub. No.: US 2008/0287406 A1
(43) Pub. Date: Nov. 20, 2008

(54) Compositions and methods for treating medical conditions

(76) Inventor: Jan N. Lessem, Natick, MA (US)

Correspondence Address: CLARK & ELBING LLP, 101 FEDERAL STREET, BOSTON, MA, 02110, US

(21) Appl. No.: 12/079,167

(22) Filed: Mar. 25, 2008

Related U.S. Application Data
(60) Provisional application No. 60/920,019, filed on Mar. 26, 2007.

(51) Int. Cl. A61K 31/57; A61P 25/00

(52) U.S. Cl. 514/171

(57) ABSTRACT

The invention features methods, compositions, and kits for the treatment of pain, pruritus, immunoinflammatory disorders, musculoskeletal disorders, and the reduction of serum C reactive protein (CRP) in a patient having a disease or conditions associated with an increased CRP level. The methods, compositions, and kits of the invention provide for a combination therapy including an SSRI and a corticosteroid.

### CROSS-REFERENCE TO RELATED APPLICATIONS

This application claims benefit from U.S. Application No. 60/920,019, filed Mar. 26, 2007, which is incorporated herein by reference.

### BACKGROUND OF THE INVENTION

The invention relates to the treatment of pain, pruritus, immunoinflammatory disorders, musculoskeletal disorders, and the reduction of serum C reactive protein (CRP) in a patient having a disease or conditions associated with an increased CRP level.

The relationship between inflammatory processes and elevation in serum CRP and proinflammatory cytokines is well known, and this relationship is observed in several disease states including cardiovascular disease (e.g., coronary artery disease, peripheral artery disease); hypertension, colon cancer, lymphoma, sarcoma, and pancreatitis. Patients with the above diseases are often treated with non-steroidal anti-inflammatory drugs (NSAIDs) such as indomethacin, ketoprofen, ibuprofen, acetylsalicyclic acid (ASA), and flubiprofen, or steroids. Steroid treatment however, often results in toxic side effects. Thus, agents or treatments that could provide the anti-inflammatory effect of steroids without the associated toxicity would be useful for reducing the level of plasma CRP and, consequently, for treating diseases and conditions associated with an elevated serum CRP level.

### SUMMARY OF THE INVENTION

The invention features methods, compositions, and kits for treating pain, pruritis, and reducing CRP levels by administering an SSRI and a corticosteroid to a patient.

Accordingly, in a first aspect, the invention features a method for reducing pain in a patient in need thereof, by administering to the patient a corticosteroid and an SSRI, wherein the corticosteroid and the SSRI are administered in amounts and for a duration that together are sufficient to reduce pain in the patient. In related embodiments of the invention, the pain that is reduced is inflammatory pain, neuropathic pain, or nociceptive pain. In related embodiments, the nociceptive pain is caused be surgery, labor, sprains, bone fractures, burns, bumps, bruises, injections, dental procedures, biopsies, or obstructions. In other embodiments, the inflammatory pain that is reduced in a patient is postoperative pain, post-traumatic pain, arthritic pain, or pain associated with damage to joints, muscle, and tendons. In other embodiments, the neuropathic pain that is reduced in a patient is caused trauma, surgery, herniation of an intervertebral disk, spinal cord injury, shingles, HIV/AIDS, late-stage cancer, amputation, and carpal tunnel syndrome. In other embodiments, the pain that is reduced is dysfunctional pain caused by fibromyalgia, tension type headache, irritable bowel disorders, or migraine.

The invention also features a method for treating pruritus in a patient in need thereof by administering to the patient a corticosteroid and an SSRI, wherein the corticosteroid and the SSRI are administered in amounts and for a duration that are together sufficient to treat the patient. In related embodiments, the pruritus to be treated is caused by rash, atopic eczema, wheals, stress, anxiety, UV radiation from the sun, metabolic and endocrine disorders, cancers, infection, or allergic reaction.

The invention further features a method for treating a musculoskeletal disorder by administering to a patient diagnosed with or at risk of developing a musculoskeletal disorder a corticosteroid and an SSRI, wherein the corticosteroid and the SSRI are administered in amounts and for a duration that are together sufficient to treat the patient.

The invention also provides a method of treating an immunoinflammatory disorder by administering to a patient diagnosed with or at risk of developing said immunoinflammatory disorder a corticosteroid and an SSRI, wherein the corticosteroids and the SSRI are administered in amounts and for a duration that are together sufficient to treat the patient. In a related embodiment, the musculoskeletal disorder treated is osteoarthritis.

The invention further features a method for reducing the serum C-reactive protein (CRP) level in a patient in need thereof by administering to the patient a corticosteroid and an SSRI, wherein the corticosteroid and the SSRI are administered in amounts and for a duration that together are sufficient to reduce the serum CRP level in the patient.

The invention also features a method for treating a disease or condition associated with an increased serum CRP level in a patient in need thereof by administering to the patient a corticosteroid and an SSRI, wherein the corticosteroid and the SSRI are administered in amounts and for a duration that together are sufficient to reduce the serum CRP level in the patient. In related embodiments, the disease or condition associated with an increased CRP level is selected from the group of cardiovascular disease, hypertension, colon cancer, lymphoma, and sarcoma.

For any of the above methods, the corticosteroid and the SSRI can be administered simultaneously or within 14 days of each other. In related embodiments of all the above methods, the corticosteroid and the SSRI are formulated in a single composition (e.g., for topical or systemic administration). In a related embodiment of all the above aspects, the corticosteroid and the SSRI are formulated for oral and systemic administration.

In all the above aspects, the corticosteroid can be a low dosage. In all the above aspects, the corticosteroid can be prednisolone and the SSRI can be paroxetine. In all the above aspects, 5 mg to 50 mg or 10 mg to 20 mg paroxetine, and 1 mg to 10 mg or 2 mg to 4 mg prednisolone can be administered daily. In all the above aspects, the corticosteroid, prednisolone, can be administered from 1 to 5 mg twice daily or 2 to 4 mg once daily. In all the above aspects, the corticosteroid, prednisolone, and the SSRI, paroxetine, can be formulated in separate dosage forms.

In all the above aspects, a third agent selected from the group consisting of antibiotics, disease-modifying anti-rheumatic drugs (DMARDs), non-steroidal anti-inflammatory drugs (NSAIDs), anti-convulsants, muscle relaxants, analgesics, cannibinoids, or sedatives can be administered to the patient.

The invention further features a kit containing a corticosteroid, an SSRI, and instructions for administering the corticosteroid and the SSRI to a patient for the treatment of pain.

The invention further feathers a kit containing a corticosteroid and instructions for administering the corticosteroid with an SSRI to a patient for the treatment of pain. In a related embodiment, the invention features a kit containing an SSRI and instructions for administering the SSRI with a corticosteroid to a patient for the treatment of pain.

Another embodiment of the invention, is a kit containing a composition containing a corticosteroid and an SSRI, and instructions for administering the composition to a patient for the treatment of pain.

In all the above kits, the pain can be inflammatory pain, neuropathic pain, or nociceptive pain. In related embodiments of the above kits, the nociceptive pain to be treated is caused by surgery, labor, sprains, bone fractures, burns, bumps, bruises, injections, dental procedures, biopsies, or obstructions. In other embodiments of the above kits, the inflammatory pain to be treated is selected from postoperative pain, post-traumatic pain, arthritic pain, or pain associated with damage to joints, muscle, and tendons. In related embodiments of the above kits, the neuropathic pain to be treated is caused by trauma, surgery, herniation of an intervertebral disk, spinal cord injury, shingles, HIV/AIDS, late-stage cancer, amputation, and carpal tunnel syndrome. In other aspects of the above kits, the pain to be treated is pain caused by fibromyalgia, tension type headache, irritable bowel disorders, or migraine.

The invention further provides a kit containing a corticosteroid, an SSRI, and instructions for administering the corticosteroid and the SSRI to a patient for the treatment of pruritus.

In a related embodiment, the invention provides a kit containing a corticosteroid and instructions for administering the corticosteroid with an SSRI to a patient for the treatment of pruritus. In another embodiment, the invention features a kit containing an SSRI and instructions for administering the SSRI with a corticosteroid to a patient for the treatment of pruritus.

The invention also provides a kit containing a composition containing a corticosteroid and an SSRI, and instructions for administering the composition to a patient for the treatment of pruritus. In all the above kits, the pruritus to be treated can be caused by rash, atopic eczema, wheals, stress, anxiety, UV radiation from the sun, metabolic and endocrine disorders, cancers, infection, or allergic reaction.

The invention also features a kit containing a corticosteroid, an SSRI, and instructions for administering the corticosteroid and the SSRI to a patient having increased serum CRP level or having a disease or condition associated with an increased serum CRP level.

In a related embodiment, the invention features a kit containing a corticosteroid and instructions for administering the corticosteroid with an SSRI to a patient having an increased serum CRP level or having a disease or condition associated with an increased serum CRP level.

The invention further features a kit containing an SSRI and instructions for administering the SSRI with a corticosteroid to a patient having an increased serum CRP level or having a disease associated with an increased serum CRP level.

In a related embodiment, the invention provides a kit containing a composition containing a corticosteroid and an SSRI, and instructions for administering the composition to a patient having an increased CRP level or having a disease or condition associated with an increased CRP level. In any of the above kits, the patient has a disease or condition associated with an increased serum CPR level selected from cardiovascular disease, hypertension, colon cancer, lymphoma, and sarcoma.

In any of the above kits, the corticosteroid and the SSRI can be formulated for topical or oral administration. In all the above kits, the corticosteroid can be in a low dosage.

In related embodiments of all the above kits, the corticosteroid is prednisolone and the SSRI is paroxetine. In different embodiments of all the above kits, instructions for administering 5 mg to 50 mg or 10 mg to 20 mg paroxetine to the patient daily are provided. In related embodiments of all the above kits, instructions for administering 1 mg to 10 mg or 2 mg to 4 mg prednisolone to the patient daily are provided.

In related embodiments of all the above kits, instructions for administering to the patient a third agent selected from the group of antibiotics, disease-modifying anti-rheumatic drugs, non-steroidal anti-inflammatory drugs (NSAIDs), anti-convulsants, muscle relaxants, analgesics, cannibinoids, or sedatives are provided.

In any of the above kits, the corticosteroid, prednisolone, and the SSRI, paroxetine, and provided in two unit dosage forms for oral administration, the first unit dosage form containing from 5 mg to 50 mg paroxetine and 1 mg to 3 mg of prednisolone, and the second dosage form containing from 1 mg to 3 mg prednisolone.

In any of the above methods and kits, the dose is from 0.5 mg to 9 mg, 0.25 mg to 3 mg, 2.0 mg to 4 mg, 2.5 mg to 10 mg, 0.5 mg to 4 mg, 0.5 mg to 1.5 mg, 0.75 mg to 1.25 mg, 1.0 mg to 10 mg, 1.0 mg to 9 mg, 1.0 mg to 8.0 mg, 1.0 mg to 7.0 mg, 1.0 mg to 6.0 mg, 1.0 mg to 5.0 mg, 1.0 mg to 4.0 mg, 1.0 mg to 3.0 mg, 2.0 mg to 8 mg, 2.0 mg to 6 mg, 3.0 mg to 10 mg, 0.75 mg to 2.5 mg, or even 0.25 mg to 1.75 mg of prednisolone or an equivalent, equipotent amount of another corticosteroid.

In any of the above methods and kits, the dose is from 0.05 mg to 200 mg, 0.1 mg to 100 mg, 1 mg to 80 mg, 1 mg to 60 mg, 1 mg to 50 mg, 2.5 mg to 50 mg, 1 mg to 40 mg, 5 mg to 30 mg, 1 mg to 30 mg, 5 mg to 50 mg, 5 mg to 26 mg, 5 mg to 25 mg, 5 mg to 24 mg, 5 mg to 23 mg, 5 mg to 22 mg, 5 mg to 21 mg, 5 mg to 20 mg, 5 mg to 18 mg, 5 mg to 16 mg, 5 mg to 14 mg, 5 mg to 12 mg, 5 mg to 10 mg, 10 mg to 20 mg, or even 11 mg to 22 mg of paroxetine or an equivalent, equipotent amount of another SSRI.

In any of the above aspects, the corticosteroid can be selected from algestone, 6-alpha-fluoroprednisolone, 6-alpha-methylprednisolone, 6-alpha-methylprednisolone 21-acetate, 6-alpha-methylprednisolone 21-hemisuccinate sodium salt, 6-alpha, 9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-17-valerate, budesonide, clobetasol, clobetasol propionate, clobetasone, clocortolone, clocortolone pivalate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, flupredidene, fluprednisolone, flurandrenolide, formocortal, halcinonide, halometasone, halopredone, hyrcanoside, hydrocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methylprednisolone, methylprednisolone acetate, methylprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-21-acetate, prednisolone-21 (beta-D-glucuronide), prednisone, prednylidene, procinonide, tralonide, triamcinolone, triamcinolone acetonide, triamcinolone acetonide 21-palmitate, triamcinolone diacetate, triamcinolone hexacetonide, and wortmannin.

In any of the above aspects, the SSRI can be selected from: cericlamine, citalopram, clovoxamine, cyanodothiepin, dapoxetine, duloxetine, escitalopram, femoxetine, 4-(2-fluorophenyl)-6-methyl-2-piperazinothieno[2,3-d]pyrimidine, fluoxetine, fluvoxamine, ifoxetine, indalpine, indeloxazine, litoxetine, milnacipran, paroxetine, sertraline, venlafaxine, viqualine, or zimeldine.

By “corticosteroid” is meant any naturally occurring or synthetic compound characterized by a hydrogenated cyclopentanoperhydro-phenanthrene ring system and having immunosuppressive and/or anti-inflammatory activity. Naturally occurring corticosteroids are generally produced by the adrenal cortex. Synthetic corticosteroids may be halogenated. Examples corticosteroids are provided herein.

Corticosteroids useful in the methods, compositions, and kits of the invention include, e.g., algestone, 6-alpha-fluoroprednisolone, 6-alpha-methylprednisolone, 6-alpha-methylprednisolone 21-acetate, 6-alpha-methylprednisolone 21-hemisuccinate sodium salt, 6-alpha, 9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-17-valerate, budesonide, clobetasol, clobetasol propionate, clobetasone, clocortolone, clocortolone pivalate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, flupredidene, fluprednisolone, flurandrenolide, formocortal, halcinonide, halometasone, halopredone, hyrcanoside, hydrocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methylprednisolone, methylprednisolone acetate, methylprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-21-acetate, prednisolone-21 (beta-D-glucuronide), prednisone, prednylidene, procinonide, tralonide, triamcinolone, triamcinolone acetonide, triamcinolone acetonide 21-palmitate, triamcinolone diacetate, triamcinolone hexacetonide, and wortmannin. Particularly desirable corticosteroids are prednisolone, cortisone, dexamethasone, hydrocortisone, methylprednisolone, fluticasone, prednisone, triamcinolone, and diflorasone.

By “a disease or condition associated with an increased serum CRP level” is meant any disease or disorder in which the level of serum CRP may be elevated compared to normal controls. Typically a serum CRP level of >3 mg/L is considered elevated. Such diseases and conditions associated with an increased serum CRP level include cardiovascular disease (e.g., coronary artery disease, peripheral artery disease); hypertension; colon cancer; lymphoma; sarcoma; and pancreatitis.

By “immunoinflammatory disorder” is meant to encompass a variety of conditions, including autoimmune diseases, proliferative skin diseases, and inflammatory dermatoses. Immunoinflammatory disorders result in the destruction of healthy tissue by an inflammatory process, deregulation of the immune system, and unwanted proliferation of cells. Examples of immunoinflammatory disorders are acne vulgaris; acute respiratory distress syndrome; Addison's disease; allergic rhinitis; allergic intraocular inflammatory diseases, ANCA-associated small-vessel vasculitis; ankylosing spondylitis; arthritis, asthma; atherosclerosis; atopic dermatitis; autoimmune hemolytic anemia; autoimmune hepatitis; Behcet's disease; Bell's palsy; bullous pemphigoid; cerebral ischaemia; chronic obstructive pulmonary disease; cirrhosis; Cogan's syndrome; contact dermatitis; COPD; Crohn's disease; Cushing's syndrome; dermatomyositis; diabetes mellitus; discoid lupus erythematosus; eosinophilic fasciitis; erythema nodosum; exfoliative dermatitis; fibromyalgia; focal glomerulosclerosis; giant cell arteritis; gout; gouty arthritis; graft-versus-host disease; hand eczema; Henoch-Schonlein purpura; herpes gestationis; hirsutism; idiopathic cerato-scleritis; idiopathic pulmonary fibrosis; idiopathic thrombocytopenic purpura; inflammatory bowel or gastrointestinal disorders, inflammatory dermatoses; lichen planus; lupus nephritis; lymphomatous tracheobronchitis; macular edema; multiple sclerosis; myasthenia gravis; myositis; osteoarthritis; pancreatitis; pemphigoid gestationis; pemphigus vulgaris; polyarteritis nodosa; polymyalgia rheumatica; pruritus scroti; pruritis/inflammation, psoriasis; psoriatic arthritis; rheumatoid arthritis; relapsing polychondritis; rosacea caused by sarcoidosis; rosacea caused by scleroderma; rosacea caused by Sweet's syndrome; rosacea caused by systemic lupus erythematosus; rosacea caused by urticaria; rosacea caused by zoster-associated pain; sarcoidosis; scleroderma; segmental glomerulosclerosis; septic shock syndrome; shoulder tendinitis or bursitis; Sjogren's syndrome; Still's disease; stroke-induced brain cell death; Sweet's disease; systemic lupus erythematosus; systemic sclerosis; Takayasu's arteritis; temporal arteritis; toxic epidermal necrolysis; tuberculosis; type-1 diabetes; ulcerative colitis; uveitis; vasculitis; and Wegener's granulomatosis. “Non-dermal inflammatory disorders” include, for example, rheumatoid arthritis, inflammatory bowel disease, asthma, and chronic obstructive pulmonary disease.

“Dermal inflammatory disorders” or “inflammatory dermatoses” include, for example, psoriasis, acute febrile neutrophilic dermatosis, eczema (e.g., asteatotic eczema, dyshidrotic eczema, vesicular palmoplantar eczema), balanitis circumscripta plasmacellularis, balanoposthitis, Behcet's disease, erythema annulare centrifugum, erythema dyschromicum perstans, erythema multiforme, granuloma annulare, lichen nitidus, lichen planus, lichen sclerosus et atrophicus, lichen simplex chronicus, lichen spinulosus, nummular dermatitis, pyoderma gangrenosum, sarcoidosis, subcorneal pustular dermatosis, urticaria, and transient acantholytic dermatosis.

“Non-dermal inflammatory disorders” include, for example, rheumatoid arthritis, inflammatory bowel disease, asthma, and chronic obstructive pulmonary disease.

By “proliferative skin disease” is meant a benign or malignant disease that is characterized by accelerated cell division in the epidermis or dermis. Examples of proliferative skin diseases are psoriasis, atopic dermatitis, non-specific dermatitis, primary irritant contact dermatitis, allergic contact dermatitis, basal and squamous cell carcinomas of the skin, lamellar ichthyosis, epidermolytic hyperkeratosis, premalignant keratosis, acne, and seborrheic dermatitis.

By “musculoskeletal disorder” is meant an immune system-related disorder of the muscles, ligaments, bones, joints, cartilage, or other connective tissue. Among the most commonly occurring musculoskeletal disorders are various forms of arthritis, e.g., osteoarthritis, rheumatoid arthritis, juvenile rheumatoid arthritis, and gout. Other musculoskeletal disorders include acquired hyperostosis syndrome, acromegaly, ankylosing spondylitis, Behcet's disease, bone diseases, bursitis, cartilage diseases, chronic fatigue syndrome, compartment syndromes, congenital hypothyroidism, congenital myopathies, dentigerous cyst, dermatomyositis, diffuse idiopathic skeletal hyperostosis, Dupuytren's contracture, eosinophilia-myalgia syndrome, fasciitis, Felty's syndrome, fibromyalgia, hallux valgus, infectious arthritis, joint diseases, Kabuki make-up syndrome, Legg-Perthes disease, lupus, Lyme disease, Melas syndrome, metabolic bone diseases, mitochondrial myopathies, mixed connective tissue disease, muscular diseases, muscular dystrophies, musculoskeletal abnormalities, musculoskeletal diseases, myositis, myositis ossificans, necrotizing fasciitis, neurogenic arthropathy, osteitis deformans, osteochondritis, osteomalacia, osteomyelitis, osteonecrosis, osteoporosis, Paget's disease, Pierre Robin syndrome, polymyalgia rheumatica, polymyositis, postpoliomyelitis syndrome, pseudogout, psoriatric arthritis, reactive arthritis, Reiter disease, relapsing polychondritis, renal osteodystrophy, rhabdomyolysis, rheumatic diseases, rheumatic fever, scleroderma, Sever's disease (calceneal apophysitis), Sjögren's syndrome, spinal diseases, spinal stenosis, Still's disease, synovitis, temporomandibular joint disorders, tendinopathy, tennis elbow, tenosynovitis, Tietze's syndrome, and Wegener's granulomatosis.

As will be appreciated by one skilled in the art, a particular disease, disorder, or condition may be characterized as being both musculoskeletal and immunoinflammatory. An example of such a disease is osteoarthritis.

By a “low dosage” is meant at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of corticosteroid formulated for administration by inhalation will differ from a low dosage of corticosteroid formulated for oral administration.

By “non-steroidal anti-inflammatory drug” or “NSAID” is meant a non-steroidal agent that prevents or diminishes inflammation. NSAIDs include naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, tolmetin, and COX-2 inhibitors such as rofecoxib, celecoxib, valdecoxib, or lumiracoxib.

The term “pain” is used herein in the broadest sense and refers to all types of pain, including acute and chronic pain, such as nociceptive pain, e.g. somatic pain and visceral pain; inflammatory pain, dysfunctional pain, neuropathic pain, e.g., centrally generated pain and peripherally generated pain, migraine, and cancer pain.

The term “nociceptive pain” is used to include all pain caused by noxious stimuli that threaten to or actually injure body tissues, including, without limitation, by a cut, bruise, bone fracture, crush injury, burn, and the like. Pain receptors for tissue injury (nociceptors) are located mostly in the skin or in the internal organs.

The term “somatic pain” is used to refer to pain arising from bone, joint, muscle, skin, or connective tissue. This type of pain is typically well localized.

The term “visceral pain” is used herein to refer to pain arising from visceral organs, such as the respiratory, gastrointestinal tract and pancreas, the urinary tract and reproductive organs. Visceral pain includes pain caused by tumor involvement of the organ capsule. Another type of visceral pain, which is typically caused by obstruction of hollow viscus, is characterized by intermittent cramping and poorly localized pain. Visceral pain may be associated with inflammation as in cystitis or reflux esophagitis.

The term “inflammatory pain” includes pain associates with active inflammation that may be caused by trauma, surgery, infection and autoimmune diseases.

The term “neuropathic pain” is used herein to refer to pain originating from abnormal processing of sensory input by the peripheral or central nervous system consequent on a lesion to these systems.

By “patient” is meant any animal (e.g., a human). Other animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds.

The term “pharmaceutically acceptable salt” represents those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the compounds of the invention, or separately by reacting the free base function with a suitable organic acid. Representative acid addition salts include acetate, ascorbate, aspartate, benzoate, citrate, digluconate, fumarate, glucoheptonate, glycerophosphate, hemisulfate, heptonate, hexanoate, hydrobromide, hydrochloride, hydroiodide, lactate, malate, maleate, malonate, mesylate, oxalate, phosphate, succinate, sulfate, tartrate, thiocyanate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

By “SSRI” is meant any member of the class of compounds that (i) inhibit the uptake of serotonin by neurons of the central nervous system, (ii) have an inhibition constant (Ki) of 10 nM or less, and (iii) a selectivity for serotonin over norepinephrine (i.e., the ratio of Ki(norepinephrine) over Ki(serotonin)) of greater than 100. Typically, SSRIs are administered in dosages of greater than 10 mg per day when used as antidepressants.

SSRIs include cericlamine (e.g., cericlamine hydrochloride); citalopram (e.g., citalopram hydrobromide); clovoxamine; cyanodothiepin; dapoxetine; duloxetine, escitalopram (escitalopram oxalate); femoxetine (e.g., femoxetine hydrochloride); 4-(2-fluorophenyl)-6-methyl-2-piperazinothieno[2,3-d]pyrimidine, fluoxetine (e.g., fluoxetine hydrochloride); fluvoxamine (e.g., fluvoxamine maleate); ifoxetine; indalpine (e.g., indalpine hydrochloride); indeloxazine (e.g., indeloxazine hydrochloride); litoxetine; milnacipran (e.g., minlacipran hydrochloride); paroxetine (e.g., paroxetine hydrochloride hemihydrate; paroxetine maleate; paroxetine mesylate); sertraline (e.g., sertraline hydrochloride); tametraline hydrochloride; venlafaxine, viqualine; and zimeldine (e.g., zimeldine hydrochloride).

By “sustained release” or “controlled release” is meant that the therapeutically active component is released from the formulation at a controlled rate such that therapeutically beneficial blood levels (but below toxic levels) of the component are maintained over an extended period of time ranging from e.g., about 12 to about 24 hours, thus, providing, for example, a 12 hour or a 24 hour dosage form.

By “systemic administration” is meant all nondermal routes of administration, and specifically excludes topical and transdermal routes of administration.

As used herein, the term “treating” refers to administering a pharmaceutical composition for prophylactic and/or therapeutic purposes. To “prevent disease” refers to prophylactic treatment of a subject who is not yet ill, but who is susceptible to, or otherwise at risk of, a particular disease. To “treat disease” or use for “therapeutic treatment” refers to administering treatment to a subject already suffering from a disease to improve or stabilize the subject's condition. Thus, in the claims and embodiments, treating is the administration to a subject either for therapeutic or prophylactic purposes.

By “an amount sufficient” is meant the amount of a compound, in a combination of the invention, required to treat or prevent a disease or condition in a clinically relevant manner. A sufficient amount of an active compound used to practice the present invention for therapeutic treatment of particular diseases and conditions caused varies depending upon the manner of administration, the age, body weight, and general health of the patient. Ultimately, the prescribers will decide the appropriate amount and dosage regimen.

Compounds useful in the invention include those described herein in any of their pharmaceutically acceptable forms, including isomers such as diastereomers and enantiomers, salts, esters, amides, thioesters, solvates, and polymorphs thereof, as well as racemic mixtures and pure isomers of the compounds described herein. As an example, by “prednisolone” is meant the free base as well as any pharmaceutically acceptable salt thereof (e.g., prednisolone acetate).

Compounds useful in the invention may also be isotopically labeled compounds. Useful isotopes include hydrogen, carbon, nitrogen, oxygen, phosphorous, fluorine, and chlorine, (e.g., ²H, ³H, ¹³C, ¹⁴C, ¹⁵N, ¹⁸O, ¹⁷O, ³¹P, ³²P, ³⁵S, ¹⁸F, and ³⁶Cl). Isotopically-labeled compounds can be prepared by synthesizing a compound using a readily available isotopically-labeled reagent in place of a non-isotopically-labeled reagent.

Other features and advantages of the invention will be apparent from the following detailed description, the drawings, and the claims.

### DETAILED DESCRIPTION

The invention provides methods, compositions, and kits for the treatment of pain, pruritis, immunoinflammatory disorders, musculoskeletal disorders, and to reduce the serum CRP level in a patient or treat a patient having a disease or condition associated with an increased serum CRP level in a patient by co-administration of a corticosteroid and an SSRI to a patient in need thereof. The co-administration of a corticosteroid and an SSRI can be an effective treatment for a disease or condition associated with an increased serum CRP level. When co-administered a corticosteroid and an SSRI can act synergistically to treat pain, pruritis, and diseases or conditions associated with an increased serum CRP level, and thereby allow lower dosages of one or both therapeutic agents to be administered, relative to the dosages required when the corticosteroid and the SSRI are administered alone for these same conditions. Alternatively, the co-administration of a corticosteroid and an SSRI can reduce side effects normally observed in patients treated with corticosteroids and SSRIs alone, even when the corticosteroid and SSRI are co-administered at dosages normally administrated when given alone.

Accordingly, the invention provides a combination method for the treatment of pain, pruritis, immunoinflammatory diseases, musculoskeletal diseases, and diseases associated with an increased serum CRP level, which differs from the previously known therapeutic strategies for treatment of these conditions. This combination method involves co-administering a corticosteroid and an SSRI each in amounts and for a duration that together are sufficient to reduce the serum CRP level in a patient or treat a disease associated with an increased serum CRP level.

The invention is described in greater detail below.

### Measurement of CRP Levels

CRP is an acute phase response protein whose production is stimulated by cytokines, particularly interleukin-6 (IL-6). The relationship between inflammatory processes and elevation in plasma CRP and pro-inflammatory cytokines is well known. Typically a serum CRP level of >3 mg/L is considered elevated. Serum CRP levels can be measured by standard techniques known in the art, e.g., ELISA assays (Kamiya Biomedical Co., Seattle, Wash.).

### Corticosteroids

Corticosteroids that are useful in the methods, compositions, and kits of this invention are selected from the class of selective glucocorticosteroid receptor agonists (SEGRAs) including, without limitation, 11-alpha, 17-alpha, 21-trihydroxypregn-4-ene-3,20-dione; 11-beta, 16-alpha, 17,21-tetrahydroxypregn-4-ene-3,20-dione; 11-beta, 16-alpha, 17,21-tetrahydroxypregn-1,4-diene-3,20-dione; 11-beta, 17-alpha, 21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha, 20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortivazol; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone 21-hemisuccinate; prednisolone acetate; prednisolone farnesylate; prednisolone hemisuccinate; prednisolone-21(beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin.

Standard recommended dosages for various steroid/disease combinations are provided in Table 1, below.

TABLE 1
Standard Recommended Corticosteroid Dosages

| Indication | Route | Drug | Dose | Schedule |
|---|---|---|---|---|
| Psoriasis | oral | Prednisolone | 7.5-60 mg | per day or divided b.i.d. |
| | oral | Prednisone | 7.5-60 mg | per day or divided b.i.d. |
| Asthma | inhaled | beclomethasone dipropionate | 42 μg/puff) | 4-8 puffs b.i.d. |
| | inhaled | Budesonide | (200 μg/inhalation) | 1-2 inhalations b.i.d. |
| | inhaled | Flunisolide | (250 μg/puff) | 2-4 puffs b.i.d. |
| | inhaled | fluticasone propionate | (44, 110 or 220 μg/puff) | 2-4 puffs b.i.d. |
| | inhaled | triamcinolone acetonide | (100 μg/puff) | 2-4 puffs b.i.d. |
| COPD | oral | Prednisone | 30-40 mg | per day |
| Crohn's disease | oral | Budesonide | 9 mg | per day |
| Ulcerative colitis | oral | Prednisone | 40-60 mg | per day |
| | oral | Hydrocortisone | 300 mg (IV) | per day |
| | oral | Methylprednisolone | 40-60 mg | per day |
| Rheumatoid arthritis | oral | Prednisone | 10 mg | per day |

Other standard recommended dosages for corticosteroids are provided, e.g., in the Merck Manual of Diagnosis & Therapy (17th Ed. M H Beers et al., Merck & Co.) and Physicians' Desk Reference 2003 (57^{th} Ed. Medical Economics Staff et al., Medical Economics Co., 2002). In one embodiment, the dosage of corticosteroid administered is a dosage equivalent to a prednisolone dosage, as defined herein. For example, a low dosage of a corticosteroid may be considered as the dosage equivalent to a low dosage of prednisolone. Two or more corticosteroids can be administered in the same treatment.

Equivalent potency in clinical dosing is well known. Information relating to equivalent steroid dosing may be found in the British National Formulary (BNF), 37 Mar. 1999, the content of which is incorporated herein by reference.

The BNF guidelines are included in Table 2 below. More specifically, Table 2 provides doses of steroids equivalent to 5 mg of prednisolone and equivalent to 1 mg of prednisolone when administered in a manner according to this invention.

TABLE 2
Equivalent Dose to Prednisolone

| | Equal to 5 mg | | Equal to 1 mg | |
|---|---|---|---|---|
| Drug | prednisolone | | prednisolone | |
| betamethasone | 750 | μg | 150 | μg |
| cortisone acetate | 25 | mg | 5 | mg |
| deflazacort | 6 | mg | 1.2 | mg |
| dexamethasone | 750 | μg | 150 | μg |
| hydrocortisone | 20 | mg | 4 | mg |
| methyl prednisone | 4 | mg | 0.8 | mg |
| triamcinolone | 4 | mg | 0.8 | mg |

It is also known (BNF 37 Mar. 1999) from clinical dosing equivalence that doses of triamcinolone, fluticasone and budesonide are broadly similar in nasal administration (110 μg, 100 μg and 200 μg).

When the combinations of the invention are used for treatment in conjunction with corticosteroids, it is possible to reduce the dosage of the individual components substantially to a point significantly below the dosages which would be required to achieve the same effects by administering corticosteroids or an SSRI alone or by administering a combination of corticosteroids and an SSRI. For example, in an SSRI/corticosteroid combination, reduced dosages of the SSRI or the corticosteroid, in comparison with dosages appropriate for administration of either compound alone, may be effective in treating a disease associated with an increased serum CRP level or reducing serum CRP levels in a patient in need thereof. Two or more corticosteroids can be administered in the same treatment.

### Selective Serotonin Reuptake Inhibitors

The methods, compositions, and kits of the invention employ an SSRI, or a structural or functional analog thereof. Suitable SSRIs include cericlamine (e.g., cericlamine hydrochloride); citalopram (e.g., citalopram hydrobromide); clovoxamine; cyanodothiepin; dapoxetine; escitalopram (escitalopram oxalate); femoxetine (e.g., femoxetine hydrochloride); fluoxetine (e.g., fluoxetine hydrochloride); fluvoxamine (e.g., fluvoxamine maleate); ifoxetine; indalpine (e.g., indalpine hydrochloride); indeloxazine (e.g., indeloxazine hydrochloride); litoxetine; milnacipran (e.g., minlacipran hydrochloride); paroxetine (e.g., paroxetine hydrochloride hemihydrate; paroxetine maleate; paroxetine mesylate); sertraline (e.g., sertraline hydrochloride); tametraline hydrochloride; viqualine; and zimeldine (e.g., zimeldine hydrochloride).

Cericlamine

Cericlamine has the following structure:

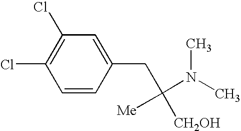

Structural analogs of cericlamine are those having the formula:

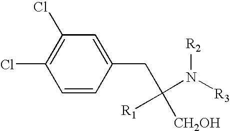

as well as pharmaceutically acceptable salts thereof, wherein R₁ is a C₁-C₄ alkyl and R₂ is H or C₁₋₄ alkyl, R₃ is H, C₁₋₄ alkyl, C₂₋₄ alkenyl, phenylalkyl or cycloalkylalkyl with 3 to 6 cyclic carbon atoms, alkanoyl, phenylalkanoyl or cycloalkylcarbonyl having 3 to 6 cyclic carbon atoms, or R₂ and R₃ form, together with the nitrogen atom to which they are linked, a heterocycle saturated with 5 to 7 chain links which can have, as the second heteroatom not directly connected to the nitrogen atom, an oxygen, a sulphur or a nitrogen, the latter nitrogen heteroatom possibly carrying a C₂₋₄ alkyl.

Exemplary cericlamine structural analogs are 2-methyl-2-amino-3-(3,4-dichlorophenyl)-propanol, 2-pentyl-2-amino-3-(3,4-dichlorophenyl)-propanol, 2-methyl-2-methylamino-3-(3,4-dichlorophenyl)-propanol, 2-methyl-2-dimethylamino-3-(3,4-dichlorophenyl)-propanol, and pharmaceutically acceptable salts of any thereof.

Citalopram

Citalopram has the following structure:

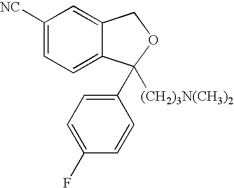

Structural analogs of citalopram are those having the formula:

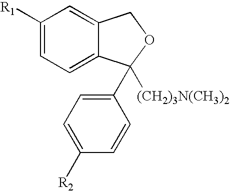

as well as pharmaceutically acceptable salts thereof, wherein each of R₁ and R₂ is independently selected from the group consisting of bromo, chloro, fluoro, trifluoromethyl, cyano and R—CO—, wherein R is C₁₋₄ alkyl.

Exemplary citalopram structural analogs (which are thus SSRI structural analogs according to the invention) are 1-(4′-fluorophenyl)-1-(3-dimethylaminopropyl)-5-bromophthalane; 1-(4′-chlorophenyl)-1-(3-dimethylaminopropyl)-5-chlorophthalane; 1-(4′-bromophenyl)-1-(3-dimethylaminopropyl)-5-chlorophthalane; 1-(4′-fluorophenyl)-1-(3-dimethylaminopropyl)-5-chlorophthalane; 1-(4′-chlorophenyl)-1-(3-dimethylaminopropyl)-5-trifluoromethyl-phthalane; 1-(4′-bromophenyl)-1-(3-dimethylaminopropyl)-5-trifluoromethyl-phthalane; 1-(4′-fluorophenyl)-1-(3-dimethylaminopropyl)-5-trifluoromethyl-phthalane; 1-(4′-fluorophenyl)-1-(3-dimethylaminopropyl)-5-fluorophthalane; 1-(4′-chlorophenyl)-1-(3-dimethylaminopropyl)-5-fluorophthalane; 1-(4′-chlorophenyl)-1-(3-dimethylaminopropyl)-5-phthalancarbonitrile; 1-(4′-fluorophenyl)-1-(3-dimethylaminopropyl)-5-phthalancarbonitrile; 1-(4′-cyanophenyl)-1-(3-dimethylaminopropyl)-5-phthalancarbonitrile; 1-(4′-cyanophenyl)-1-(3-dimethylaminopropyl)-5-chlorophthalane; 1-(4′-cyanophenyl)-1-(3-dimethylaminopropyl)-5-trifluoromethylphthalane; 1-(4′-fluorophenyl)-1-(3-dimethylaminopropyl)-5-phthalancarbonitrile; 1-(4′-chlorophenyl)-1-(3-dimethylaminopropyl)-5-ionylphthalane; 1-(4-(chlorophenyl)-1-(3-dimethylaminopropyl)-5-propionylphthalane; and pharmaceutically acceptable salts of any thereof.

Clovoxamine

Clovoxamine has the following structure:

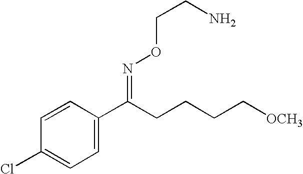

Structural analogs of clovoxamine are those having the formula:

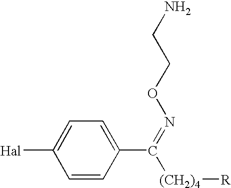

as well as pharmaceutically acceptable salts thereof, wherein Hal is a chloro, bromo, or fluoro group and R is a cyano, methoxy, ethoxy, methoxymethyl, ethoxymethyl, methoxyethoxy, or cyanomethyl group.

Exemplary clovoxamine structural analogs are 4′-chloro-5-ethoxyvalerophenone O-(2-aminoethyl)oxime; 4′-chloro-5-(2-methoxyethoxy)valerophenone O-(2-aminoethyl)oxime; 4′-chloro-6-methoxycaprophenone O-(2-aminoethyl)oxime; 4′-chloro-6-ethoxycaprophenone O-(2-aminoethyl)oxime; 4′-bromo-5-(2-methoxyethoxy)valerophenone O-(2-aminoethyl)oxime; 4′-bromo-5-methoxyvalerophenone O-(2-aminoethyl)oxime; 4′-chloro-6-cyanocaprophenone O-(2-aminoethyl)oxime; 4′-chloro-5-cyanovalerophenone O-(2-aminoethyl)oxime; 4′-bromo-5-cyanovalerophenone O-(2-aminoethyl)oxime; and pharmaceutically acceptable salts of any thereof.

Femoxetine

Femoxetine has the following structure:

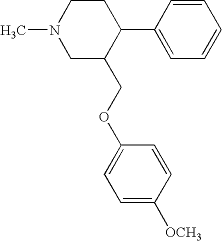

Structural analogs of femoxetine are those having the formula:

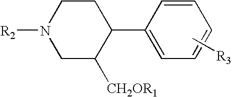

wherein R₁ represents a C₁₋₄ alkyl or C₂₋₄ alkynyl group, or a phenyl group optionally substituted by C₁₋₄ alkyl, C₁₋₄ alkylthio, C₁₋₄ alkoxy, bromo, chloro, fluoro, nitro, acylamino, methylsulfonyl, methylenedioxy, or tetrahydronaphthyl, R₂ represents a C₁₋₄ alkyl or C₂₋₄ alkynyl group, and R₃ represents hydrogen, C₁₋₄ alkyl, C₁₋₄alkoxy, trifluoroalkyl, hydroxy, bromo, chloro, fluoro, methylthio, or aralkyloxy.

Exemplary femoxetine structural analogs are disclosed in Examples 7-67 of U.S. Pat. No. 3,912,743, hereby incorporated by reference.

Fluoxetine

Fluoxetine has the following structure:

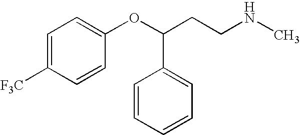

Structural analogs of fluoxetine are those compounds having the formula:

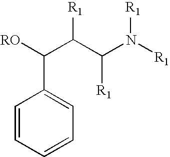

as well as pharmaceutically acceptable salts thereof, wherein each R₁ is independently hydrogen or methyl; R is naphthyl or

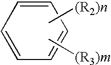

wherein each of R₂ and R₃ is, independently, bromo, chloro, fluoro, trifluoromethyl, C₁₋₄ alkyl, C₁₋₃ alkoxy or C₃₋₄ alkenyl; and each of n and m is, independently, 0, 1 or 2. When R is naphthyl, it can be either α-naphthyl or β-naphthyl.

Exemplary fluoxetine structural analogs are 3-(p-isopropoxyphenoxy)-3-phenylpropylamine methanesulfonate, N,N-dimethyl 3-(3′,4′-dimethoxyphenoxy)-3-phenylpropylamine p-hydroxybenzoate, N,N-dimethyl 3-(α-naphthoxy)-3-phenylpropylamine bromide, N,N-dimethyl 3-(β-naphthoxy)-3-phenyl-1-methylpropylamine iodide, 3-(2′-methyl-4′,5′-dichlorophenoxy)-3-phenylpropylamine nitrate, 3-(p-t-butylphenoxy)-3-phenylpropylamine glutarate, N-methyl 3-(2′-chloro-p-tolyloxy)-3-phenyl-1-methylpropylamine lactate, 3-(2′,4′-dichlorophenoxy)-3-phenyl-2-methylpropylamine citrate, N,N-dimethyl 3-(m-anisyloxy)-3-phenyl-1-methylpropylamine maleate, N-methyl 3-(p-tolyloxy)-3-phenylpropylamine sulfate, N,N-dimethyl 3-(2′,4′-difluorophenoxy)-3-phenylpropylamine 2,4-dinitrobenzoate, 3-(o-ethylphenoxy)-3-phenylpropylamine dihydrogen phosphate, N-methyl 3-(2′-chloro-4′-isopropylphenoxy)-3-phenyl-2-methylpropylamine maleate, N,N-dimethyl 3-(2′-alkyl-4′-fluorophenoxy)-3-phenyl-propylamine succinate, N,N-dimethyl 3-(o-isopropoxyphenoxy)-3-phenyl-propylamine phenylacetate, N,N-dimethyl 3-(o-bromophenoxy)-3-phenyl-propylamine β-phenylpropionate, N-methyl 3-(p-iodophenoxy)-3-phenyl-propylamine propiolate, and N-methyl 3-(3-n-propylphenoxy)-3-phenyl-propylamine decanoate.

Fluvoxamine

Fluvoxamine has the following structure:

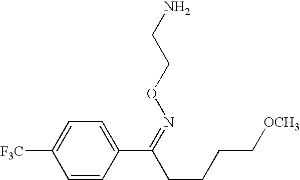

Structural analogs of fluvoxamine are those having the formula:

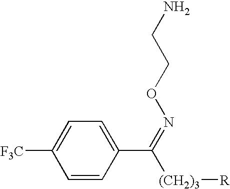

as well as pharmaceutically acceptable salts thereof, wherein R is cyano, cyanomethyl, methoxymethyl, or ethoxymethyl.

Indalpine

Indalpine has the following structure:

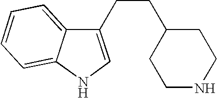

Structural analogs of indalpine are those having the formula:

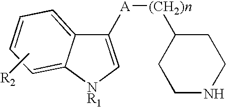

or pharmaceutically acceptable salts thereof, wherein R₁ is a hydrogen atom, a C₁-C₄ alkyl group, or an aralkyl group of which the alkyl has 1 or 2 carbon atoms, R₂ is hydrogen, C₁₋₄ alkyl, C₁₋₄ alkoxy or C₁₋₄ alkylthio, chloro, bromo, fluoro, trifluoromethyl, nitro, hydroxy, or amino, the latter optionally substituted by one or two C₁₋₄ alkyl groups, an acyl group or a C₁₋₄alkylsulfonyl group; A represents —CO or —CH₂— group; and n is 0, 1 or 2.

Exemplary indalpine structural analogs are indolyl-3 (piperidyl-4 methyl) ketone; (methoxy-5-indolyl-3) (piperidyl-4 methyl) ketone; (chloro-5-indolyl-3) (piperidyl-4 methyl) ketone; (indolyl-3)-1(piperidyl-4)-3 propanone, indolyl-3 piperidyl-4 ketone; (methyl-1 indolyl-3) (piperidyl-4 methyl) ketone, (benzyl-1 indolyl-3) (piperidyl-4 methyl) ketone; [(methoxy-5 indolyl-3)-2 ethyl]-piperidine, [(methyl-1 indolyl-3)-2 ethyl]-4-piperidine; [(indolyl-3)-2 ethyl]-4 piperidine; (indolyl-3 methyl)-4 piperidine, [(chloro-5 indolyl-3)-2 ethyl]-4 piperidine; [(indolyl-b 3)-3 propyl]-4 piperidine; [(benzyl-1 indolyl-3)-2 ethyl]-4 piperidine; and pharmaceutically acceptable salts of any thereof.

Indeloxazine

Indeloxezine has the following structure:

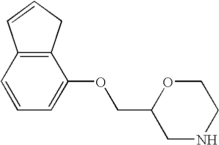

Structural analogs of indeloxazine are those having the formula:

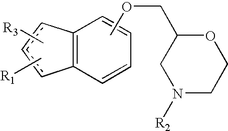

and pharmaceutically acceptable salts thereof, wherein R₁ and R₃ each represents hydrogen, C₁₋₄ alkyl, or phenyl; R₂ represents hydrogen, C₁₋₄ alkyl, C₄₋₇ cycloalkyl, phenyl, or benzyl; one of the dotted lines means a single bond and the other means a double bond, or the tautomeric mixtures thereof.

Exemplary indeloxazine structural analogs are 2-(7-indenyloxymethyl)-4-isopropylmorpholine; 4-butyl-2-(7-indenyloxymethyl)morpholine; 2-(7-indenyloxymethyl)-4-methylmorpholine; 4-ethyl-2-(7-indenyloxymethyl)morpholine, 2-(7-indenyloxymethyl)-morpholine; 2-(7-indenyloxymethyl)-4-propylmorpholine; 4-cyclohexyl-2-(7-indenyloxymethyl)morpholine; 4-benzyl-2-(7-indenyloxymethyl)-morpholine; 2-(7-indenyloxymethyl)-4-phenylmorpholine; 2-(4-indenyloxymethyl)morpholine; 2-(3-methyl-7-indenyloxymethyl)-morpholine; 4-isopropyl-2-(3-methyl-7-indenyloxymethyl)morpholine; 4-isopropyl-2-(3-methyl-4-indenyloxymethyl)morpholine; 4-isopropyl-2-(3-methyl-5-indenyloxymethyl)morpholine; 4-isopropyl-2-(1-methyl-3-phenyl-6-indenyloxymethyl)morpholine; 2-(5-indenyloxymethyl)-4-isopropyl-morpholine, 2-(6-indenyloxymethyl)-4-isopropylmorpholine; and 4-isopropyl-2-(3-phenyl-6-indenyloxymethyl)morpholine; as well as pharmaceutically acceptable salts of any thereof.

Milnacipram

Milnacipram has the following structure:

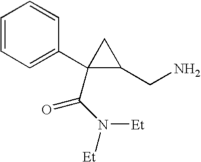

Structural analogs of milnacipram are those having the formula:

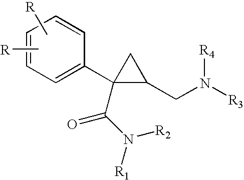

as well as pharmaceutically acceptable salts thereof, wherein each R, independently, represents hydrogen, bromo, chloro, fluoro, C₁₋₄ alkyl, C₁₋₄ alkoxy, hydroxy, nitro or amino; each of R₁ and R₂, independently, represents hydrogen, C₁₋₄ alkyl, C₆₋₁₂ aryl or C₇₋₁₄ alkylaryl, optionally substituted, preferably in para position, by bromo, chloro, or fluoro, or R₁ and R₂ together form a heterocycle having 5 or 6 members with the adjacent nitrogen atoms; R₃ and R₄ represent hydrogen or a C₁₋₄ alkyl group or R₃ and R₄ form with the adjacent nitrogen atom a heterocycle having 5 or 6 members, optionally containing an additional heteroatom selected from nitrogen, sulphur, and oxygen.

Exemplary milnacipram structural analogs are 1-phenyl 1-aminocarbonyl 2-dimethylaminomethyl cyclopropane; 1-phenyl 1-dimethylaminocarbonyl 2-dimethylaminomethyl cyclopropane; 1-phenyl 1-ethylaminocarbonyl 2-dimethylaminomethyl cyclopropane; 1-phenyl 1-diethylaminocarbonyl 2-aminomethyl cyclopropane; 1-phenyl 2-dimethylaminomethyl N-(4′-chlorophenyl)cyclopropane carboxamide; 1-phenyl 2-dimethylaminomethyl N-(4′-chlorobenzyl)cyclopropane carboxamide; 1-phenyl 2-dimethylaminomethyl N-(2-phenylethyl)cyclopropane carboxamide; (3,4-dichloro-1-phenyl) 2-dimethylaminomethyl N,N-dimethylcyclopropane carboxamide; 1-phenyl 1-pyrrolidinocarbonyl 2-morpholinomethyl cyclopropane; 1-p-chlorophenyl 1-aminocarbonyl 2-aminomethyl cyclopropane; 1-orthochlorophenyl 1-aminocarbonyl 2-dimethylaminomethyl cyclopropane; 1-p-hydroxyphenyl 1-aminocarbonyl 2-dimethylaminomethyl cyclopropane; 1-p-nitrophenyl 1-dimethylaminocarbonyl 2-dimethylaminomethyl cyclopropane; 1-p-aminophenyl 1-dimethylaminocarbonyl 2-dimethylaminomethyl cyclopropane; 1-p-tolyl 1-methylaminocarbonyl 2-dimethylaminomethyl cyclopropane; 1-p-methoxyphenyl 1-aminomethylcarbonyl 2-aminomethyl cyclopropane; and pharmaceutically acceptable salts of any thereof.

Paroxetine

Paroxetine has the following structure:

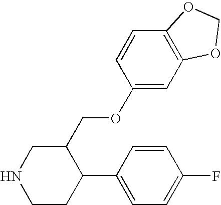

Structural analogs of paroxetine are those having the formula:

| Name | Structure |
|---|---|
| (1R, 4S) Sertraline Hydrochloride | 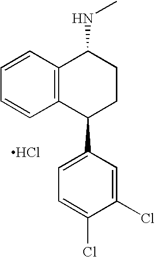 |
| (1S, 4R) Sertraline Hydrochloride | 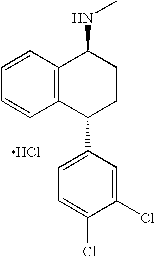 |
| Sertraline B-Ring Para-Phenoxy | 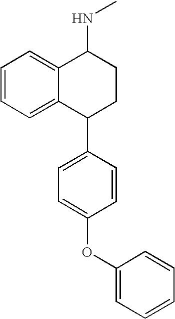 |
| Sertraline B-Ring Ortho-Methoxy | 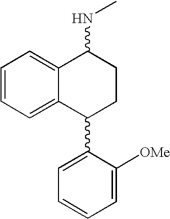 |
| 1R,4R Sertraline Enantiomer | 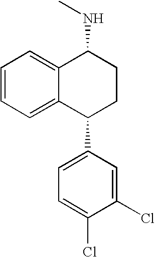 |
| Sertraline Sulfonamide | 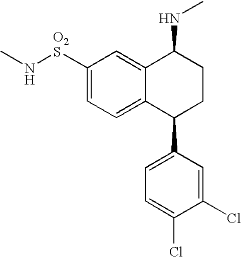 |
| Nitro Sertraline | 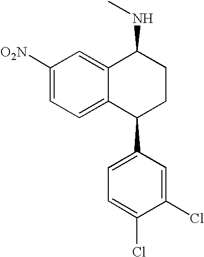 |
| Sertraline Aniline | 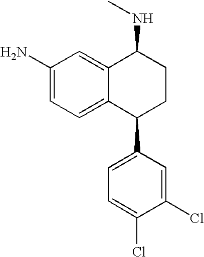 |
| Sertraline Reverse Sulfonamide (CH2linker) | 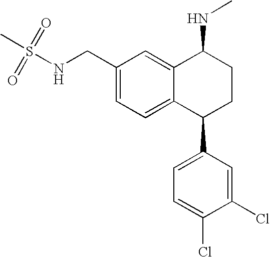 |
| UK-416244 | 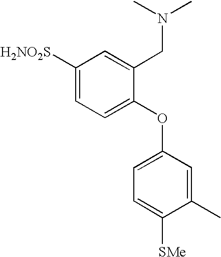 |
| (1R,4R)-Desmethyl Sertraline | 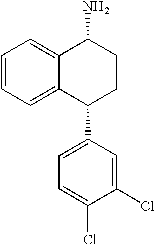 |
| Sertraline A-Ring Methyl Ester | 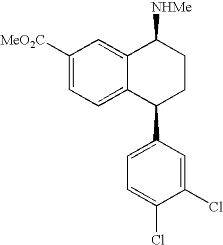 |
| rac-cis-N-Desmethyl Sertraline,Hydrochloride | 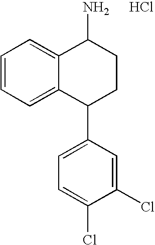 |
| Dimethyl Sertraline Reverse Sulfonamide | 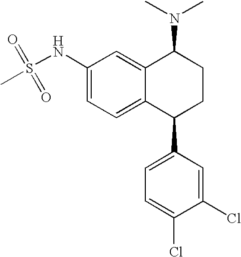 |
| Sertraline N,N-Dimethylsulfonamide | 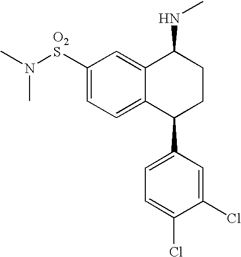 |
| Sertraline A-Ring Ethanol | 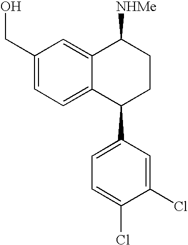 |
| Sertraline-CME | 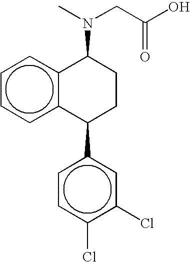 |
| (1S,4S)-Desmethyl Sertraline,Hydrochloride | 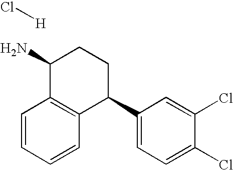 |
| Sertraline Iodide | 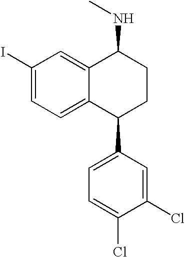 |
| 1-Des(methylamine)-1-oxo-2-(R,S)-hydroxy Sertraline | 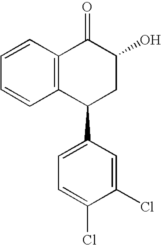 |
| Sertraline Nitrile | 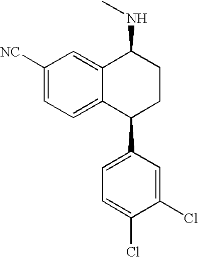 |
| Sertraline Hydrochloride | 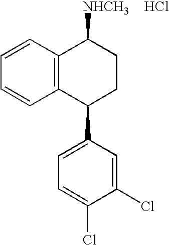 |
| N,N-Dimethyl Sertraline B-Ring Para-Trifluoromethane | 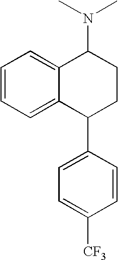 |
| Sertraline Sulfonamide NH2 | 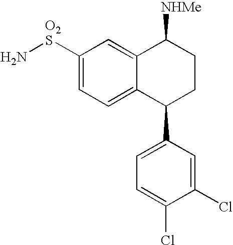 |
| Sertraline (Reverse) Methanesulfonamide | 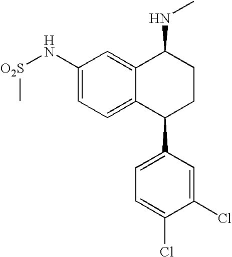 |
| Sertraline A-Ring Carboxylic Acid | 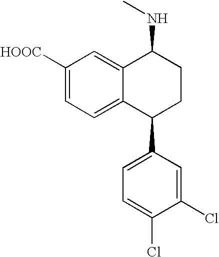 |
| Sertraline Sulfonamide Ethanol | 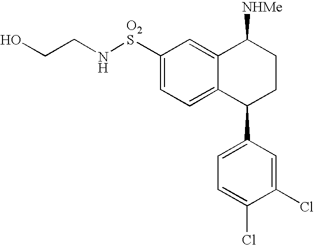 |
| Sertraline B-Ring Para-Trifluoromethane | 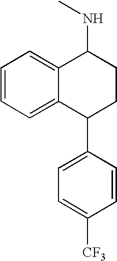 |
| N,N-Dimethyl Sertraline | 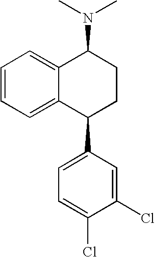 |

Particularly useful are the following compounds, in either the (1S)-enantiomeric or (1S)(1R) racemic forms, and their pharmaceutically acceptable salts: cis-N-methyl-4-(3,4-dichlorophenyl)-1,2,3,4-tetrahydro-1-naphthalenamine; cis-N-methyl-4-(4-bromophenyl)-1,2,3,4-tetrahydro-1-naphthalenamine; cis-N-methyl-4-(4-chlorophenyl)-1,2,3,4-tetrahydro-1-naphthalenamine; cis-N-methyl-4-(3-trifluoromethyl-phenyl)-1,2,3,4-tetrahydro-1-naphthalenamine; cis-N-methyl-4-(3-trifluoromethyl-4-chlorophenyl)-1,2,3,4-tetrahydro-1-naphthalenamine; cis-N,N-dimethyl-4-(4-chlorophenyl)-1,2,3,4-tetrahydro-1-naphthalenamine; cis-N,N-dimethyl-4-(3-trifluoromethyl-phenyl)-1,2,3,4-tetrahydro-1-naphthalenamine; and cis-N-methyl-4-(4-chlorophenyl)-7-chloro-1,2,3,4-tetrahydro-1-naphthalenamine. Of interest also is the (1R)-enantiomer of cis-N-methyl-4-(3,4-dichlorophenyl)-1,2,3,4-tetrahydro-1-naphthalenamine.

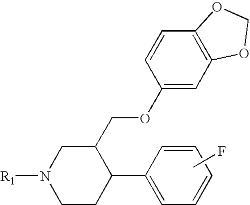

and pharmaceutically acceptable salts thereof, wherein R₁ represents hydrogen or a C₁₋₄ alkyl group, and the fluorine atom may be in any of the available positions.

### Sertraline

Sertraline has the following structure:

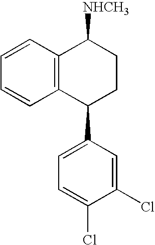

Structural analogs of sertraline are those having the formula:

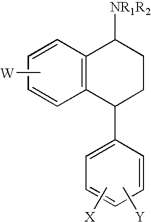

wherein R₁ is selected from the group consisting of hydrogen and C₁₋₄ alkyl; R₂ is C₁₋₄ alkyl; X and Y are each selected from the group consisting of hydrogen, fluoro, chloro, bromo, trifluoromethyl, C₁₋₃ alkoxy, and cyano; and W is selected from the group consisting of hydrogen, fluoro, chloro, bromo, trifluoromethyl and C₁₋₃ alkoxy. Preferred sertraline analogs are in the cis-isomeric configuration. The term “cis-isomeric” refers to the relative orientation of the NR₁R₂ and phenyl moieties on the cyclohexene ring (i.e. they are both oriented on the same side of the ring). Because both the 1- and 4-carbons are asymmetrically substituted, each cis-compound has two optically active enantiomeric forms denoted (with reference to the 1-carbon) as the cis-(1R) and cis-(1S) enantiomers. Sertraline analogs are also described in U.S. Pat. No. 4,536,518. Other related compounds include (S,S)—N-desmethylsertraline, rac-cis-N-desmethylsertraline, (1S,4S)-desmethyl sertraline, 1-des (methylamine)-1-oxo-2-(R,S)-hydroxy sertraline, (1R,4R)-desmethyl sertraline, sertraline sulfonamide, sertraline (reverse) methanesulfonamide, 1R,4R sertraline enantiomer, N,N-dimethyl sertraline, nitro sertraline, sertraline aniline, sertraline iodide, sertraline sulfonamide NH₂, sertraline sulfonamide ethanol, sertraline nitrile, sertraline-CME, dimethyl sertraline reverse sulfonamide, sertraline reverse sulfonamide (CH₂ linker), sertraline B-ring ortho methoxy, sertraline A-ring methyl ester, sertraline A-ring ethanol, sertraline N,N-dimethylsulfonamide, sertraline A ring carboxylic acid, sertraline B-ring para-phenoxy, sertraline B-ring para-trifluoromethane, N,N-dimethyl sertraline B-Ring para-trifluoromethane, and UK-416244. Structures of these analogs are shown below.

Zimeldine

Zimeldine has the following structure:

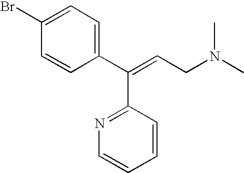

Structural analogs of zimeldine are those compounds having the formula:

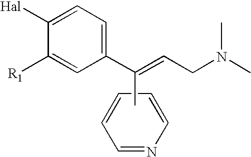

and pharmaceutically acceptable salts thereof, wherein the pyridine nucleus is bound in ortho-, meta- or para-position to the adjacent carbon atom and where R₁ is selected from the group consisting of H, chloro, fluoro, and bromo.

Exemplary zimeldine analogs are (e)- and (z)-3-(4′-bromophenyl-3-(2″-pyridyl)-dimethylallylamine; 3-(4′-bromophenyl)-3-(3″-pyridyl)-dimethylallylamine; 3-(4′-bromophenyl)-3-(4″-pyridyl)-dimethylallylamine; and pharmaceutically acceptable salts of any thereof.

Structural analogs of any of the above SSRIs are considered herein to be SSRI analogs and thus may be employed in any of the methods, compositions, and kits of the invention.

Metabolites

Pharmacologically active metabolites of any of the foregoing SSRIs can also be used in the methods, compositions, and kits of the invention. Exemplary metabolites are didesmethylcitalopram, desmethylcitalopram, desmethylsertraline, and norfluoxetine.

Analogs

Functional analogs of SSRIs can also be used in the methods, compositions, and kits of the invention. Exemplary SSRI functional analogs are provided below. One class of SSRI analogs are SNRIs (selective serotonin norepinephrine reuptake inhibitors), which include venlafaxine and duloxetine.

Venlafaxine

Venlafaxine has the following structure:

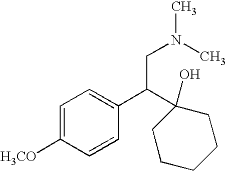

Structural analogs of venlafaxine are those compounds having the formula:

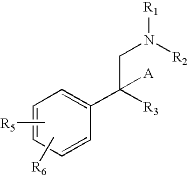

as well as pharmaceutically acceptable salts thereof, wherein A is a moiety of the formula:

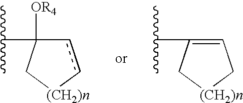

where the dotted line represents optional unsaturation; R₁ is hydrogen or alkyl; R₂ is C₁₋₄ alkyl; R₄ is hydrogen, C₁₋₄ alkyl, formyl or alkanoyl; R₃ is hydrogen or C₁₋₄ alkyl; R₅ and R₆ are, independently, hydrogen, hydroxyl, C₁₋₄ alkyl, C₁₋₄ alkoxy, C₁₋₄ alkanoyloxy, cyano, nitro, alkylmercapto, amino, C₁₋₄ alkylamino, dialkylamino, C₁₋₄ alkanamido, halo, trifluoromethyl or, taken together, methylenedioxy; and n is 0, 1, 2, 3 or 4.

Duloxetine

Duloxetine has the following structure:

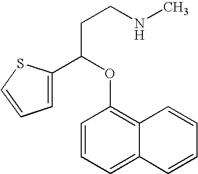

Structural analogs of duloxetine are those compounds described by the formula disclosed in U.S. Pat. No. 4,956,388, hereby incorporated by reference.

Other SSRI analogs are 1,2,3,4-tetrahydro-N-methyl-4-phenyl-1-naphthylamine hydrochloride; 1,2,3,4-tetrahydro-N-methyl-4-phenyl-(E)-1-naphthylamine hydrochloride; N,N-dimethyl-1-phenyl-1-phthalanpropylamine hydrochloride; gamma-(4-(trifluoromethyl)phenoxy)-benzenepropanamine hydrochloride; BP 554; CP 53261; O-desmethylvenlafaxine; WY 45,818; WY 45,881; N-(3-fluoropropyl)paroxetine; and Lu 19005.

Standard Recommended Dosages

Standard recommended dosages for exemplary SSRIs are provided in Table 2, below. Other standard dosages are provided, e.g., in the Merck Manual of Diagnosis & Therapy (17th Ed. M H Beers et al., Merck & Co.) and Physicians' Desk Reference 2003 (57^{th} Ed. Medical Economics Staff et al., Medical Economics Co., 2002).

TABLE 3

| Compound | Standard Dose | |
|---|---|---|
| Fluoxetine | 20-80 | mg/day |
| Sertraline | 50-200 | mg/day |
| Paroxetine | 20-50 | mg/day |
| Fluvoxamine | 50-300 | mg/day |
| Citalopram | 10-80 | mg qid |
| Escitalopram | 10 | mg qid |

Non-Steroidal Anti-Inflammatory Drugs (NSAIDs)

If desired, the co-administration of an SSRI and a corticosteroid of the invention may be administered in conjunction with one or more non-steroidal anti-inflammatory drugs (NSAIDs), such as naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin.

When an SSRI and a corticosteroid are administered in combination with acetylsalicylic acid, it is desirable that the combination be effective in decreasing serum CRP level or treating a disease or disorder associated with increased serum CRP level. Accordingly, the combination of an SSRI (e.g., paroxetine) and a corticosteroid (e.g., prednisolone) in combination with acetylsalicylic acid or its analogs may be more effective in treating diseases or disorders associated with an increased serum CRP level.

Acetylsalicylic acid, also known by trade name aspirin, is an acetyl derivative of salicylic acid and has the following structural formula:

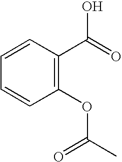

Aspirin is useful in the relief of headache and muscle and joint aches. Aspirin is also effective in reducing fever, inflammation, and swelling, and thus has been used for treatment of, e.g., osteoarthritis. Thus, the combination of a tetra-substituted pyrimidopyrimidine or analog thereof (e.g., dipyridamole, or an adenosine activity upregulator) and acetylsalicylic acid (aspirin) or analog thereof can also be administered to enhance the treatment or prevention of the disorders mentioned herein.

An NSAID may be administered in conjunction with any one of the combinations described in this application. For example, a patient suffering from a disease or disorder associated with an increased serum CRP level may be initially treated with a combination of an SSRI and a corticosteroid, and the patient may further be treated with an NSAID.

Dosage amounts of acetylsalicylic acid are known to those skilled in medical arts, and generally range from about 70 mg to about 350 mg per day. When a lower or a higher dose of aspirin is needed, a formulation containing dipyridamole and aspirin may contain 0-25 mg, 25-50 mg, 50-70 mg, 70-75 mg, 75-80 mg, 80-85 mg, 85-90 mg, 90-95 mg, 95-100 mg, 100-150 mg, 150-160 mg, 160-250 mg, 250-300 mg, 300-350 mg, or 350-1000 mg of aspirin.

When the combinations of the invention are used for treatment in conjunction with NSAIDs, it is possible to reduce the dosage of the individual components substantially to a point significantly below the dosages which would be required to achieve the same effects by administering NSAIDs (e.g., acetylsalicylic acid) alone.

Two or more NSAIDs can be administered in the same treatment.

### Disease Modifying Anti-Rheumatic Drugs

Disease modifying anti-rheumatic drugs (DMARDs) can be used in the methods, compositions, and kits of the invention. DMARDs are a class of anti-inflammatory drugs. Examples of DMARDs known in the art include, but are not limited to anakinra, auranofin, aurothioglucose, azathioprine, chlorambucil, cyclophosphamide, cyclosporine, D-penicillamine, gold sodium thiomalate (injectable gold), hydroxychloroquine, leflunomide, methotrexate, minocycline, mycophenol mofetil, or sulfasalazine.

Methotrexate is an example of a DMARD that can be used in one embodiment of the combination treatment method of this invention. Methotrexate, also known as Amethopterin, RHEUMATREX® (Lederle Pharmaceutical), or FOLEX® (Aventis), is an antimetabolite that competitively and reversibly inhibits dihydrofolate reductase (DHFR), an enzyme that is part of the folate synthesis metabolic pathway. The affinity of methotrexate for DHFR is about one thousand-fold that of folate for DHFR, which catalyses the conversion of dihydrofolate to the active tetrahydrofolate. Folic acid is needed for the de novo synthesis of the nucleoside thymidine, required for DNA synthesis. Methotrexate is therefore capable of inhibiting the synthesis of DNA, RNA, and thymidylates. Targeting the S-phase of the cell cycle, methotrexate has a greater negative effect on rapidly dividing cells. As a result, methotrexate has been prescribed for treating a number of medical conditions including certain cancers, severe psoriasis, and inflammatory arthritic diseases.

The chemical name for methotrexate is N-[4-[[(2,4-diamino-6-pteridinyl)methyl]methylamino]benzoyl]-L-glutamic acid, although it is commonly present in the form of a sodium salt in pharmaceutical compositions and its amount in such compositions is determined by equivalence to the free acid. Therefore, when a composition is said to contain 10 mg of methotrexate, a greater weight of a sodium salt of methotrexate may be present in the composition. Methotrexate is a generic drug that has been in use for many years and is commercially available through various suppliers. For instance, methotrexate is manufactured and marketed by both Pfizer and Wyeth.

Dosage amounts of DMARDs are known to those skilled in medical arts, and generally range from about 0.1 to 3,000 mg per dose one or more times per week (e.g., 2, 3, 4, 5, 6, or 7 or more times per week), 0.1 to 2,500 mg per dose per week, 0.1 to 2,000 mg per dose per week, 0.1 to 1,000 mg per dose per week, 0.1 to 750 mg per dose per week, 0.1 to 500 mg per dose per week, 0.1 to 250 mg per dose per week, or 0.1 to 100 mg per dose per week.

### Therapy

The invention features methods for treating pain, pruritis, and for reducing serum CRP level in a patient or treating a patient having a disease or disorder associated with an increased serum CRP level. The reduction in serum CRP level is achieved by administering one or more SSRI in combination with one or more steroid. While the examples describe the combination of a single SSRI and a single steroid, it is understood that the combination of multiple agents is often desirable. In addition, one more SSRIs and one or more corticosteroids may be co-administered with a tertiary agent (e.g., antibiotics, DMARDs, or NSAIDs).

Therapy according to the invention may be performed alone or in conjunction with another therapy and may be provided at home, the doctor's office, a clinic, a hospital's outpatient department, or a hospital. The duration of the therapy depends on the type of disease or disorder being treated, the age and condition of the patient, the stage and type of the patient's disease, and how the patient responds to the treatment. Additionally, a person having a greater risk of developing an inflammatory disease (e.g., a person who is undergoing age-related hormonal changes) may receive treatment to inhibit or delay the onset of symptoms.

In particular embodiments of any of the methods of the invention, the compounds are administered simultaneously or within fourteen days, ten days, five days, 24 hours, or 1 hour of each other in amounts sufficient to treat the patient. The compounds may be formulated together as a single composition, or may be formulated and administered separately (e.g., separate dosage forms). One or both compounds may be administered in a low dosage or in a high dosage, each of which is defined herein. It may be desirable to administer to the patient other compounds in addition to one more corticosteroid and one or more SSRI, such as an NSAID (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, and tolmetin), NsIDIs (e.g., cyclosporine, tacrolimus, pimecrolimus, and ISAtx247), antibiotics, or DMARDs. Combination therapies of the invention are especially useful for the treatment of diseases or disorders associated with an increased serum CRP levels in combination with other agents that modulate the immune response to positively affect disease. Such agents include those that deplete key inflammatory cells, influence cell adhesion, or influence cytokines involved in immune response. This last category includes both agents that mimic or increase the action of anti-inflammatory cytokines such as IL-10, as well as agents inhibit the activity of pro-inflammatory cytokines such as IL-6, IL-1, IL-2, IL-12, IL-15 or TNFα. Agents that inhibit TNFα include etanercept, adelimumab, infliximab, and CDP-870. In this example (that of agents blocking the effect of TNFα), the combination therapy reduces the production of cytokines, etanercept or infliximab act on the remaining fraction of inflammatory cytokines, providing enhanced treatment. Small molecule immunodulators include, e.g., p38 MAP kinase inhibitors such as VX 702, SCIO 469, doramapimod, RO 30201195, SCIO 323, TACE inhibitors such as DPC 333, ICE inhibitors such as pranalcasan, and IMPDH inhibitors such as mycophenolate and merimepodib.

In combination therapy of the invention, the dosage and frequency of administration of each component of the combination can be controlled independently. For example, one compound may be administered three times per day, while the second compound may be administered once per day. Combination therapy may be given in on-and-off cycles that include rest periods so that the patient's body has a chance to recover from any as yet unforeseen side effects. The compounds may also be formulated together such that one administration delivers both compounds.

In the therapy, one agent (e.g., a corticosteroid) may be administered to a patient in a first treatment period, followed by administration of both the first agent and a second agent (e.g., an SSRI) during a second treatment period, followed by a third treatment period, wherein only the first agent is administered alone, wherein the first, second, and third treatment periods are within a continuous treatment regimen. The this type of treatment regimen may be repeated one or more times for a patient having an increased CRP level or having an disease or disorder associated with an increased serum CRP level.

The compounds in question may be administered orally in the form of tablets, capsules, elixirs or syrups, or rectally in the form of suppositories. Parenteral administration of the compound is suitably performed, for example, in the form of saline solutions or with the compound incorporated into liposomes. In cases where the compound in itself is not sufficiently soluble to be dissolved, a solubilizer such as ethanol can be applied. Additionally, the compositions may be formulated for epidural or intrathecal administration.

Desirably, the methods, compositions, and kits of the invention are more effective than other methods, compositions, and kits. By “more effective” is meant that a method, composition, or kit exhibits greater efficacy, is less toxic, safer, more convenient, better tolerated, or less expensive, or provides more treatment satisfaction than another method, composition, or kit with which it is being compared.

### Cotherapy

If desired, one or more additional agents may be administered in conjunction with the methods, compositions, and kits of the invention. Suitable agents include antibiotics (minocycline, penicillin, cephalosporin, tetracycline, oxytetracycline, chlortetracycline, metronidazole, chloramphenicol, streptomycin, neomycin, sulfonamides, phenolic compounds, quaternary ammonium compounds, doxycycline); antiseptics (e.g., chlorhexidine); nonsteroidal antiinflammatories (e.g., flurbiprofen, carprofen, diclofenac, fenbufen, fenclozic acid, fenoprofen, flufenamic acid, ibuprofen, indomethacin, indoprofen, ketoprofen, lonazolac, loxoprofen, meclofenamic acid, mefanamic acid, naproxen, proprionic acids, salicylic acids, sulindac, tolmetin, meloxicam, oxicams, piroxicam, tenoxicam, etodolac, and oxaprozin); tranexamic acid, allantoin; epsilon-aminocaproic acid; lysozyme; dihydrocholesterol; beta-glycyrrhetinic acid; platelet aggregation inhibitors (e.g., abciximab, aspirin, cilostazol, clopidogrel, eptifibatide, ticlopidine, or tirofiban); anticoagulants (e.g., dalteparin, danaparoid, enoxaparin, heparin, tinzaparin, or warfarin); antipyretics (e.g., acetaminophen); ticlopidine; clopidogrel; angiotensin converting enzyme inhibitors; beta blockers; pentoxifylline; cilostazol; estrogen replacement therapy; and lipid-lowering agents (e.g., cholestyramine, colestipol, nicotinic acid, gemfibrozil, probucol, ezetimibe, or statins such as atorvastatin, rosuvastatin, lovastatin simvastatin, pravastatin, cerivastatin, and fluvastatin). These agents may be administered concomitantly or within 14 days of the method of the invention. If desired, one or more of the foregoing agents is coformulated with one or more agents of the invention to form a single composition. Thus, in one embodiment, the invention features an SSRI, one of the foregoing agents, and a corticosteroid.

Osteoarthritis

The methods, compositions, and kits of the invention may be used for the treatment of osteoarthritis, or pain associated therewith. If desired, one or more agents typically used to treat osteoarthritis may be used with the corticosteroid therapy methods, compositions, and kits of the invention. Such agents include NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), NsIDIs (e.g., cyclosporine, tacrolimus, pimecrolimus, and ISAtx247), or analogs thereof. Thus, in one embodiment, the invention features the combination of an SSRI with any of the foregoing agents and a corticosteroid in the methods and kits for the treatment of osteoarthritis or pain associated therewith.

Chronic Obstructive Pulmonary Disease

In one embodiment, the methods, compositions, and kits of the invention are used for the treatment of chronic obstructive pulmonary disease (COPD). If desired, one or more agents typically used to treat COPD may be used with the corticosteroid therapy methods, compositions, and kits of the invention. Such agents include xanthines (e.g., theophylline), anticholinergic compounds (e.g., ipratropium, tiotropium), biologics, small molecule immunomodulators, and beta receptor agonists/bronchdilators (e.g., ibuterol sulfate, bitolterol mesylate, epinephrine, formoterol fumarate, isoproteronol, levalbuterol hydrochloride, metaproterenol sulfate, pirbuterol scetate, salmeterol xinafoate, and terbutaline. Thus, in one embodiment, the invention features the combination of an SSRI with a bronchodilator and a corticosteroid for treating COPD.

Psoriasis

The methods, compositions, and kits of the invention may be used for the treatment of psoriasis. If desired, one or more antipsoriatic agents typically used to treat psoriasis may be used with the corticosteroid therapy methods, compositions, and kits of the invention. Such agents include biologics (e.g., alefacept, inflixamab, adelimumab, efalizumab, etanercept, and CDP-870), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), vitamin D analogs (e.g., calcipotriene, calcipotriol), psoralens (e.g., methoxsalen), retinoids (e.g., acitretin, tazoretene), DMARDs (e.g., methotrexate), and anthralin. Thus, in one embodiment, the invention features the combination of an SSRI with an antipsoriatic agent and a corticosteroid for treating psoriasis.

Inflammatory Bowel Disease

The methods, compositions, and kits of the invention may be used for the treatment of inflammatory bowel disease. If desired, one or more agents typically used to treat inflammatory bowel disease may be used with the corticosteroid therapy methods, compositions, and kits of the invention. Such agents include biologics (e.g., inflixamab, adelimumab, and CDP-870), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate and azathioprine) and alosetron.

Thus, in one embodiment, the invention features the combination of an SSRI with any of the foregoing agents and a corticosteroid for treating inflammatory bowel disease.

Rheumatoid Arthritis

The methods, compositions, and kits of the invention may be used for the treatment of rheumatoid arthritis. If desired, one or more agents typically used to treat rheumatoid arthritis may be used with the corticosteroid therapy methods, compositions, and kits of the invention. Such agents include NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), COX-2 inhibitors (e.g., rofecoxib, celecoxib, valdecoxib, and lumiracoxib), biologics (e.g., inflixamab, adelimumab, etanercept, CDP-870, rituximab, and atlizumab), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate, leflunomide, minocycline, auranofin, gold sodium thiomalate, aurothioglucose, and azathioprine), hydroxychloroquine sulfate, and penicillamine. Thus, in one embodiment, the invention features the combination of an SSRI with any of the foregoing agents and a corticosteroid for treating rheumatoid arthritis.

Asthma

The methods, compositions, and kits of the invention may be used for the treatment of asthma. If desired, one or more agents typically used to treat asthma may be used with the corticosteroid therapy methods, compositions, and kits of the invention. Such agents include beta 2 agonists/bronchodilators/leukotriene modifiers (e.g., zafirlukast, montelukast, and zileuton), biologics (e.g., omalizumab), small molecule immunomodulators, anticholinergic compounds, xanthines, ephedrine, guaifenesin, cromolyn sodium, nedocromil sodium, and potassium iodide. Thus, in one embodiment, the invention features the combination of an SSRI with any of the foregoing agents and a corticosteroid for treating asthma.

Pain

The methods, compositions, and kits of the invention may be used for the treatment of pain (e.g., neuropathic pain or nociceptive pain). If desired, one or more agents typically used to treat pain may be used with the corticosteroid therapy methods, compositions, and kits of the invention. Such agents include NSAIDs, opioids, tricyclic antidepressants, anticonvulsants, amantadine, tramadol, oxycodone, buproprion, mexiletine, capsaicin, muscle relaxants, pregabalin, ketamide, analgesics, SSRIs, cannibinoids, sedatives, and anti-anxiety drugs.

Anticonvulsants

The anticonvulsants are used in prevention of the occurrence of epileptic seizures. The goal of an anticonvulsant is to suppress the rapid and excessive firing of neurons that start a seizure. Many anticonvulsants block sodium (Na+) channels, calcium (Ca2+) channels, AMPA receptors, or NMDA receptors. Some anticonvulsants inhibit the metabolism of GABA or increase its release.

Anti-convulsants include barbiturates (e.g., amobarbital, aprobarbital, barbital, butabarbital, butalbital, hexobarbital, methohexital, pentobarbital, secobarbital, sodium thiopental, talbutal, thiobarbital, Phenobarbital, methylphenobarbital, metharbital, barbexaclone), benzodiazepines (e.g., alprazolam, bromazepam, chlordiazepoxide, cinolazepam, clonazepam, clorazepate, diazepam, estazolam, flunitrazepam, flurazepam, halazepam, ketazolam, loprazolam, lorazepam, lormetazepam, medazepam, midazolam, nitrazepam, nordazepam, oxazepam, phenazepam, pinazepam, prazepam, quazepam, temazepam, tetrazepam, and triazolam), carboxamide (e.g., carbamazepine and oxcarbazepine), vigabatrin, progabide, and tiagabine topiramate, gabapentin, pregabalin, hydantoins (e.g., ethotoin, phenyloin, mephenyloin, and fosphenyloin), oxazolidinediones (e.g., paramethadione, trimethadione, ethadione), beclamide, primidone, pyrrolidines (e.g., brivaracetam, levetiracetam, and seletracetam), succinimides (e.g., ethosuximide, phensuximide, and mesuximide), sulfonamides (e.g., acetazolamide, sulthiame, methazolamide, and zonisamide), lamotrigine, pheneturide, phenacemide, valpromide, valnoctamide, and valproate.

Muscle Relaxants

A muscle relaxant is a drug that decreases the tone of a muscle. Muscle relaxants include methocarbamol, baclofen, carisoprodol, chlorzoxazone, cyclobenzaprine, dantrolene, metaxalone, orphenadrine, pancuronium, tizanidine, and dicyclomine.

Analgesics

Analgesics are compounds used to treat pain. Analgesics include opiods (e.g., morphine, codeine, thebaine, oxycodone, hydrocodone, dihydrocodeine, hydromorphone, oxymorphone, nicomorphine, methadone, levo-alphacetylmethadol, fentanyl, alfentanil, sufentanil, remifentanil, ketobemidone, carfentanyl, ohmefentanyl, ketobemidone, allylprodine, prodine, PEPAP, propoxyphene, dextropropoxyphene, dextromoramide, bezitramide, piritramide, pentazocine, phenazocine, buprenorphine, butorphanol, nalbufine, levorphanol, levomethorphan, dezocine, etorphine, lefetamine, tilidine, tramadol, naloxone, and naltrexone), NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), acetaminophen, and COX-2 inhibitors (e.g., rofecoxib, celecoxib, valdecoxib, and lumiracoxib).

Cannibinoids

Cannabinoids are a group of diterpene C21 compounds present in Cannabis sativa L and include a group of substances that are structurally related to THC or that bind to cannabinoid receptors. Cannibinoids include CP-55940, HU-210, SR141716, SR144528, WIN 55, 212-2, JWH-133, Nabilone, Levonantradol, Marinol, and Sativex.

Sedatives

A sedative is a substance that depresses the central nervous system (CNS), resulting in calmness, relaxation, reduction of anxiety, sleepiness, slowed breathing, slurred speech, staggering gait, poor judgment, and slow, uncertain reflexes. Sedatives include chlorpromazine, fluphenazine, haloperidol, loxapine succinate, perphenazine, prochlorperazine, thiothixene, trifluoperazine, clozapine, olanzapine, quetiapine, risperidone, ziprasidone, catnip, Kava Kava, Mandrake, valerian, chloral hydrate, diethyl ether, eszopiclone, ethchlorvynol, ethyl alcohol, gamma-hydroxybutyrate, glutethimide, meprobamate, methaqualone, methyl trichloride, methyprylon, ramelteon, zaleplon, zolpidem, and zopiclone.

Thus, in one embodiment, the invention features the combination of any of the foregoing agents and a corticosteroid for treating pain.

The pain that can be treated using the methods, compositions, and kits of the invention include pain caused as a result of neuropathy, including diabetic neuropathy, polyneuropathy, cancer pain, fibromyalgia, myofascial pain syndrome, osteoarthritis, pancreatic pain, pelvic/perineal pain, post herpetic neuralgia, rheumatoid arthritis, sciatica/lumbar radiculopathy, spinal stenosis, temporo-mandibular joint disorder, HIV pain, trigeminal neuralgia, chronic neuropathic pain, lower back pain, failed back surgery pain, back pain, post-operative pain, post physical trauma pain (including gunshot, road traffic accident, burns), cardiac pain, chest pain, pelvic pain/PID, joint pain (tendonitis, bursitis, acute arthritis), neck pain, bowel pain, phantom limb pain, obstetric pain (labour/C-Section), renal colic, acute herpes zoster pain, acute pancreatitis breakthrough pain (cancer), and dysmenorhoea/endometriosis. The methods, compositions, and kits of the invention can also be used to treat pain caused as a result of inflammatory disease, or as a result of combined inflammatory, autoimmune and neuropathic tissue damage, including rheumatoid arthritis, osteoarthritis, rheumatoid spondylitis, gouty arthritis, and other arthritic conditions, cancer, HIV, chronic pulmonary inflammatory disease, silicosis, pulmonary sarcosis, bone resorption diseases, reperfusion injury (including damage caused to organs as a consequence of reperfusion following ischaemic episodes e.g. myocardial infarcts, strokes), autoimmune damage (including multiple sclerosis, Guillam Barre Syndrome, myasthenia gravis) graft v. host rejection, allograft rejections, fever and myalgia due to infection, AIDS related complex (ARC), keloid formation, scar tissue formation, Crohn's disease, ulcerative colitis and pyresis, irritable bowel syndrome, osteoporosis, cerebral malaria and bacterial meningitis, bowel pain, cancer pain, back pain, fibromyalgia, and post-operative pain.

### Formulation of Pharmaceutical Compositions

The administration of a combination of the invention may be by any suitable means that results in treatment in a patient in need thereof. The compound may be contained in any appropriate amount in any suitable carrier substance, and is generally present in an amount of 1-95% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time period after administration, using controlled release formulations.

Administration of compounds in controlled release formulations is useful where the compound, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD50) to median effective dose (ED50)); (ii) a narrow absorption window in the gastro-intestinal tract; or (iii) a short biological half-life, so that frequent dosing during a day is required in order to sustain the plasma level at a therapeutic level.

Many strategies can be pursued to obtain controlled release in which the rate of release outweighs the rate of metabolism of the therapeutic compound. For example, controlled release can be obtained by the appropriate selection of formulation parameters and ingredients, including, e.g., appropriate controlled release compositions and coatings. Examples include single or multiple unit tablet or capsule compositions, oil solutions, suspensions, emulsions, microcapsules, microspheres, nanoparticles, patches, and liposomes.

Each compound of the combination may be formulated in a variety of ways that are known in the art. For example, the first and second agents may be formulated together or separately. Desirably, the first and second agents are formulated together for the simultaneous or near simultaneous administration of the agents.

The individually or separately formulated agents can be packaged together as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions.

The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

In one desirable embodiment, the corticosteroid is in a unit dosage form having a first dose formulated for immediate release and a second dose formulated for 2 to 10 hour delayed release. In another desirable embodiment, the corticosteroid is formulated for 2 to 10 hour sustained release. Desirably, the corticosteroid is from 1 to 30 mg of prednisolone, or an equivalent, equipotent amount of another corticosteroid. Each of these two corticosteroid formulations is described in co-pending U.S. Provisional Application No. 60/920,011, entitled “SPLIT DOSE CORTICOSTEROID THERAPY” and having Attorney Docket No. 50164/154001 (filed Mar. 26, 2007), hereby incorporated by reference.

Solid Dosage Forms for Oral Use

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

The two compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

Thus, for compositions adapted for oral use, an oral vehicle (e.g., a capsule) containing from between 0.01% to 25% (w/w) or more of an SSRI and/or corticosteroid, preferably from between 0.01% to 10% (w/w), more preferably from between 0.05% to 4% (w/w) active agent. The capsule can be taken one to four times daily, or as needed.

Performing the methods described herein, the oral vehicle containing an SSRI and/or the additional agent is preferably taken orally. For example, a capsule may be taken in the morning and one in the evening by a subject suffering from a disease or disorder associated with an increased serum CRP level.

Topical Formulations

Compositions can also be adapted for topical use with a topical vehicle containing from between 0.0001% and 25% (w/w) or more of the SSRI and between 0.001% and 25% (w/w) and more of a corticosteroid.

Inhalation

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

Dosages

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used.

Generally, when administered orally to a human, the dosage of the SSRI is normally about 0.001 mg to 200 mg per day, desirably about 1 mg to 100 mg per day, and more desirably about 5 mg to 50 mg per day. Dosages up to 200 mg per day may be necessary. For administration of the SSRI by injection, the dosage is normally about 1 mg to 250 mg per day, desirably about 5 mg to 200 mg per day, and more desirably about 10 mg to 150 mg per day. Injections are desirable given four times daily.

The dosage range for corticosteroids is wide, and patient response is variable. Generally, when systemically administered to a human, the dosage of the corticosteroid for use in combination with the SSRI is normally about 0.1 mg to 1500 mg per day, desirably about 0.5 mg to 10 mg per day, and more desirably about 0.5 mg to 5 mg per day. Dosages up to 3000 mg per day may be necessary.

The specific amounts of drugs administered depend on the specific combination of components. In a desired dose combination, the ratio of SSRI to steroid is about 50:1 by weight, more desirably about 20:1 or 10:1 by weight, and most desirably about 4:1, 2:1, or 1:1 by weight.

Administration of each drug in the combination can, independently, be one to four times daily for one day to one year, and may even be for the life of the patient. Chronic, long-term administration will be indicated in many cases.

As described above, each compound may be administered orally in the form of tablets, capsules, elixirs or syrups, or rectally in the form of suppositories, such that the drugs are absorbed into the bloodstream. Parenteral administration of a compound is suitably performed, for example, in the form of saline solutions or with the compound incorporated into liposomes. In cases where the compound in itself is not sufficiently soluble to be dissolved, a solubilizer such as ethanol can be applied.

### Pain, Function, and Fatigue Indices

In order to measure the efficacy of any of the methods, compositions, or kits of the invention, a measurement index may be used. Indices that are useful in the methods, compositions, and kits of the invention include a visual analog scale (VAS), a Likert scale, the Lequesne index, the WOMAC index, the AUSCAN index, the Piper Fatigue Scale, and the Multidimensional Assessment of Fatigue (MAF) scale, each of which is well known in the art. Such indices may be used to measure pain, function, fatigue, stiffness, tenderness, impairment in mobility, soft tissue swelling, bony swelling, or other variables.

A visual analog scale (VAS) provides a measure of a one-dimensional quantity. A VAS generally utilizes a representation of distance, such as a picture of a line with hash marks drawn at regular distance intervals, e.g., ten 1-cm intervals. For example, a patient can be asked to rank a sensation of pain by choosing the spot on the line that best corresponds to the sensation of pain, where one end of the line corresponds to “no pain” (score of 0 cm) and the other end of the line corresponds to “unbearable pain” (score of 10 cm). This procedure provides a simple and rapid approach to obtaining quantitative information about how the patient is experiencing pain. VAS scales can also be used, e.g., to measure fatigue. VAS scales and their use are described, e.g., in U.S. Pat. Nos. 6,709,406 and 6,432,937.

A Likert scale similarly provides a measure of a one-dimensional quantity. Generally, a Likert scale has discrete integer values ranging from a low value (e.g., 0, meaning no pain) to a high value (e.g., 7, meaning extreme pain). A patient experiencing pain is asked to choose a number between the low value and the high value to represent the degree of pain experienced. Likert scales can also be used, e.g., to measure fatigue. Likert scales and their use are described, e.g., in U.S. Pat. Nos. 6,623,040 and 6,766,319.

The Lequesne index and the Western Ontario and McMaster Universities (WOMAC) osteoarthritis index assess pain, function, and stiffness in the knee and hip of OA patients using self-administered questionnaires. Both knee and hip are encompassed by the WOMAC, whereas there is one Lequesne questionnaire for the knee and a separate one for the hip. These questionnaires are useful because they contain more information content in comparison with VAS or Likert. Both the WOMAC index and the Lequesne index questionnaires have been extensively validated in OA, including in surgical settings (e.g., knee and hip arthroplasty). Their metric characteristics do not differ significantly.

The AUSCAN (Australian-Canadian hand arthritis) index employs a valid, reliable, and responsive patient self-reported questionnaire. In one instance, this questionnaire contains 15 questions within three dimensions (Pain, 5 questions; Stiffness, 1 question; and Physical function, 9 questions). An AUSCAN index may utilize, e.g., a Likert or a VAS scale.

The Piper Fatigue scale is a 41-item measure of fatigue developed for research purposes and tested with oncology patients (Piper et al. (1989), The development of an instrument to measure the subjective dimension of fatigue. In S. Funk, E. Tornquist, M. Champagne, & R. Wiese (Eds.). Key aspects of comfort: Management of pain, fatigue, and nausea (pp. 199-207). New York: Springer.) The Multidimensional Assessment of Fatigue (MAF) scale, a revision of the Piper Fatigue scale, contains 15 items and measures four dimensions of fatigue: severity (#1-2), distress (#3), degree of interference in activities of daily living (#4-14), and frequency (#15), with scores ranging from 1 (no fatigue) to 50 (severe fatigue). The MAF has been validated in RA patients (Belza, J. Rheumatol. 22:639-643, 1995).

### Rheumatoid Arthritis Indices

In order to measure the efficacy of any of the methods, compositions, or kits of the invention, a measurement index may be used. Indices that are useful in the methods, compositions, and kits of the invention include the ACR-20/50/70 and the disease activity score (DAS).

ACR-20/50/70

ACR-20/50/70 is a widely accepted composite index of improvement in RA proposed by the American College of Rheumatology (ACR). ACR-20/50/70 refers to a composite improvement of 20%, 50% or 70% in swollen joint count, tender joint count, and 3 or more of the following 5 measures: patient's own global assessment of RA disease activity; physician's global assessment of disease activity; patient's own assessment of pain due to R; acute-phase reactant (CRP); and patient's self-addressed disability (Health Assessment Questionnaire).

DAS28

The disease activity score (DAS) is a combined index that was developed in Nijmegen in the 1980s to measure the disease activity in patients with RA. It has been extensively validated for its use in clinical trials in combination with the European League Against Rheumatism (EULAR) response criteria. To calculate the DAS28, the number of swollen joints and tender joints should be assessed using 28-joint counts, the CRP levels should be measured in mg/L, and the patients general health (GH) or global disease activity measured on a Visual Analog Scale (VAS) of 100 mm must be obtained. Using this data, the DAS28 using CRP (mg/L) can be calculated using the following formula:

DAS28=0.56*sqrt(tender28)+0.28*sqrt(swollen28)+0.36*ln(CRP+1)+0.014*GH+0.96

The DAS28 provides a number between 0 and 10 indicating the current activity of RA in the patient. A DAS above 5.1 means high disease activity, and below 3.2 indicates low activity. Remission is achieved by a DAS28 lower than 2.6.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### EXAMPLES

### Study Protocol

We conducted a 14 week blinded, randomized study including a split dose corticosteroid therapy or placebo, with regular CRP and inflammatory cytokine measurements. The study population had active rheumatoid arthritis. The subject must otherwise have been in good general health.

During the study, subjects attended the following study visits:

Screening Visit
Day 1 (Baseline Visit)—Start of Run-In Period
Day 14 (±2 days)—End of Run-In Period/Start of Treatment Period
Day 42 (±2 days)
Day 70 (±2 days)—End of Treatment Period/Start of Withdrawal Period
Day 98 (±2 days)—End of Study

All eligible subjects received DMARD therapy in a stable dose. Subjects were evaluated for study eligibility at the Screening visit, which was conducted within 14 days before the first dose of study drug. The subject provided written informed consent to participate in the study before any Screening laboratory samples were collected or evaluations performed.

All subjects were given 3 mg prednisolone alone for 2 weeks (Run-In Period). Subjects were then randomized into treatment groups to additionally receive 10 mg paroxetine, 20 mg paroxetine or placebo tablets for 8 weeks (Combination Treatment Period). The powered study ended after these first 10 weeks. All subjects continued in an un-powered part of the study for an additional 4 weeks (Withdrawal Period). In this portion of the study, half of the subjects on 10 mg or 20 mg paroxetine plus 3 mg prednisolone stopped taking paroxetine, and instead took 3 mg prednisolone plus placebo, while the other half stopped taking prednisolone, and instead took either dose of paroxetine plus placebo. Similarly, half of the subjects on prednisolone plus placebo stopped the prednisolone, and instead took two placebo tablets, while the other half continued the regimen of 3 mg prednisolone plus placebo. A schematic of the study design is shown below.

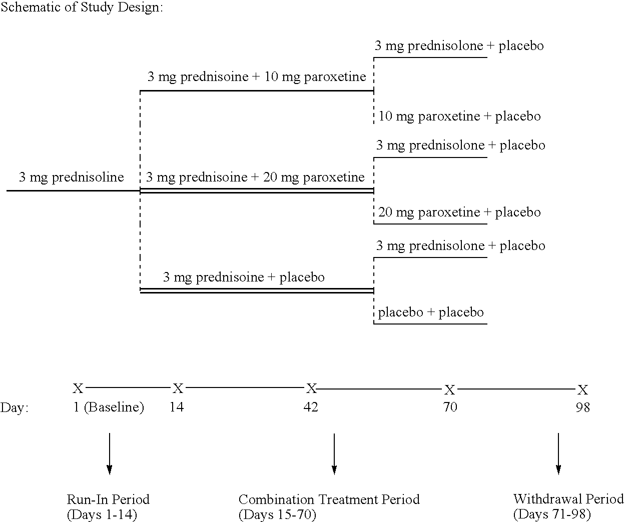

The drugs were blister packed as follows:

Steroid Run-In Period (Days 1-14)

| | 8 am | 1 pm |
|---|---|---|
| All subjects | 1 mg prednisolone | 1 mg prednisolone |
| | 1 mg prednisolone | |

Combination Treatment Period (Days 15-70)

| | 8 am | 1 pm |
|---|---|---|
| Dose Level 1 | 10 mg paroxetine | 1 mg prednisolone |
| | 1 mg prednisolone | 1 mg prednisolone |
| Dose Level 2 | 20 mg paroxetine | 1 mg prednisolone |
| | 1 mg prednisolone | 1 mg prednisolone |
| Placebo | placebo | 1 mg prednisolone |
| | 1 mg prednisolone | 1 mg prednisolone |

Withdrawal Period (Days 71-98)

| | 8 am | 1 pm |
|---|---|---|
| Dose Level 1a | placebo | 1 mg prednisolone |
| | 1 mg prednisolone | 1 mg prednisolone |
| Dose Level 1b | 10 mg paroxetine | placebo |
| | placebo | placebo |
| Dose Level 2a | placebo | 1 mg prednisolone |
| | 1 mg prednisolone | 1 mg prednisolone |
| Dose Level 2b | 20 mg paroxetine | placebo |
| | placebo | placebo |
| Placebo A | placebo | 1 mg prednisolone |
| | 1 mg prednisolone | 1 mg prednisolone |
| Placebo B | placebo | placebo |
| | placebo | placebo |

The results of the study are shown in Tables 4-11. While the paroxetine/prednisolone combinations were not statistically significant when compared to 3 mg prednisolone as measured by ACR20 at day 70, these combinations did achieve statistical significant when compared to 3 mg prednisolone alone as measured by ACR20 and ACR50 at earlier time points.

### Study Results

TABLE 4
Summary of American College of Rheumatology (ACR) Responses
for Prednisolone Vs. Combined Paroxetine Groups
ITT Population (Missing Values = Non-Responder)

| | | | | 3 mg |
|---|---|---|---|---|
| | | | 3 mg | Prednisolone |
| | | | Prednisolone | 10-20 mg |
| | | Response | Placebo | Paroxetine |
| ACR | Visit | from Baseline | (N = 69) | (N = 140) |
| ACR20 | Day 14 | Yes | 16 (23) | 32 (23) |
| | | No | 53 (77) | 108 (77) |
| | | Pvalue | | 0.9573 |
| | Day 42 | Yes | 25 (36) | 73 (52) |
| | | No | 44 (64) | 67 (48) |
| | | Pvalue | | 0.0302 |
| | Day 70 | Yes | 30 (43) | 58 (41) |
| | | No | 39 (57) | 82 (59) |
| | | Pvalue | | 0.7778 |
| ACR50 | Day 14 | Yes | 5 (7) | 5 (4) |
| | | No | 64 (93) | 135 (96) |
| | | Pvalue | | 0.3036* |
| | Day 42 | Yes | 9 (13) | 31 (22) |
| | | No | 60 (87) | 109 (78) |
| | | Pvalue | | 0.1158 |
| | Day 70 | Yes | 14 (20) | 30 (21) |
| | | No | 55 (80) | 110 (79) |
| | | Pvalue | | 0.8494 |
| ACR70 | Day 14 | Yes | 3 (4) | 0 |
| | | No | 66 (96) | 140 (100) |
| | | Pvalue | | 0.0349* |
| | Day 42 | Yes | 2 (3) | 10 (7) |
| | | No | 67 (97) | 130 (93) |
| | | Pvalue | | 0.3442* |
| | Day 70 | Yes | 4 (6) | 10 (7) |
| | | No | 65 (94) | 130 (93) |
| | | Pvalue | | 1.0000* |

Note:
p-value derived from a Chi-Square Test determining differences in proportions between treatment group and placebo.
*If any expected cell count is less than 5 Fisher Exact test used.

TABLE 5
Summary of American College of Rheumatology (ACR) Responses for
Prednisolone Vs. Each Paroxetine Group
ITT Population (Missing Values = Non-Responder)

| | | | | 3 mg | 3 mg |
|---|---|---|---|---|---|
| | | | 3 mg | Prednisolone | Prednisolone |
| | | Response | Prednisolone | 10 mg | 20 mg |
| | | from | Placebo | Paroxetine | Paroxetine |
| ACR | Visit | Baseline | (N = 69) | (N = 71) | (N = 69) |
| ACR20 | Day 14 | Yes | 16 (23) | 14 (20) | 18 (26) |
| | | No | 53 (77) | 57 (80) | 51 (74) |
| | | Pvalue | | 0.6169 | 0.6928 |
| | Day 42 | Yes | 25 (36) | 35 (49) | 38 (55) |
| | | No | 44 (64) | 36 (51) | 31 (45) |
| | | Pvalue | | 0.1184 | 0.0263 |
| | Day 70 | Yes | 30 (43) | 27 (38) | 31 (45) |
| | | No | 39 (57) | 44 (62) | 38 (55) |
| | | Pvalue | | 0.5117 | 0.8639 |
| ACR50 | Day 14 | Yes | 5 (7) | 3 (4) | 2 (3) |
| | | No | 64 (93) | 68 (96) | 67 (97) |
| | | Pvalue | | 0.4902* | 0.4409* |
| | Day 42 | Yes | 9 (13) | 9 (13) | 22 (32) |
| | | No | 60 (87) | 62 (87) | 47 (68) |
| | | Pvalue | | 0.9482 | 0.0080 |
| | Day 70 | Yes | 14 (20) | 11 (15) | 19 (28) |
| | | No | 55 (80) | 60 (85) | 50 (72) |
| | | Pvalue | | 0.4588 | 0.3184 |
| ACR70 | Day 14 | Yes | 3 (4) | 0 | 0 |
| | | No | 66 (96) | 71 (100) | 69 (100) |
| | | Pvalue | | 0.1171* | 0.2445* |
| | Day 42 | Yes | 2 (3) | 3 (4) | 7 (10) |
| | | No | 67 (97) | 68 (96) | 62 (90) |
| | | Pvalue | | 1.0000* | 0.1652* |
| | Day 70 | Yes | 4 (6) | 4 (6) | 6 (9) |
| | | No | 65 (94) | 67 (94) | 63 (91) |
| | | Pvalue | | 1.0000* | 0.5114 |

Note:
p-value derived from a Chi-Square Test determining differences in proportions between treatment group and placebo.
*If any expected cell count is less than 5 Fisher Exact test used.

TABLE 6
Summary of CRP for Prednisolone Vs. Combined Paroxetine Groups
ITT Population (LOCF)

| | 3 mg Prednisolone + Placebo | | | 3 mg Prednisolone + 10-20 mg Paroxetine | | |
|---|---|---|---|---|---|---|
| | (N = 69) | | | (N = 140) | | |
| | | Change from | % Change from | | Change from | % Change from |
| Visit | Value | baseline | baseline | Value | baseline | baseline |
| Baseline | | | | | | |
| N | 69 | | | 140 | | |
| Mean | 18.83 | | | 12.39 | | |
| (Std Dev) | (23.749) | | | (15.780) | | |
| Median | 8.90 | | | 6.55 | | |
| Min, Max | 0.3, 135.3 | | | 0.2, 91.6 | | |
| Day 14 | | | | | | |
| N | 69 | 69 | 69 | 140 | 140 | 140 |
| Mean | 11.42 | −7.40 | 7.11 | 8.36 | −4.03 | −16.96 |
| (Std Dev) | (17.413) | (20.149) | (215.970) | (11.624) | (9.748) | (80.108) |
| Median | 6.20 | −1.70 | −32.26 | 4.45 | −1.45 | −36.28 |
| Min, Max | 0.3, 107.3 | −83.2, 77.9 | −95.8, 1700.0 | 0.0, 70.0 | −63.8, 29.0 | −100.0, 522.2 |
| p-value | | | | | 0.6263 | 0.4161 |
| Day 42 | | | | | | |
| N | 69 | 69 | 69 | 140 | 140 | 140 |
| Mean | 10.22 | −8.61 | −17.67 | 9.89 | −2.50 | 3.67 |
| (Std Dev) | (12.443) | (18.009) | (58.589) | (14.263) | (9.573) | (209.843) |
| Median | 5.40 | −1.00 | −23.53 | 4.25 | −1.05 | −28.11 |
| Min, Max | 0.3, 70.6 | −81.4, 21.5 | −92.5, 187.3 | 0.1, 72.0 | −35.8, 55.3 | −97.6, 2340.0 |
| p-value | | | | | 0.2687 | 0.7120 |
| Day 70 | | | | | | |
| N | 69 | 69 | 69 | 140 | 140 | 140 |
| Mean | 9.88 | −8.95 | −13.66 | 10.62 | −1.77 | 6.02 |
| (Std Dev) | (11.956) | (18.996) | (64.905) | (14.625) | (8.443) | (106.063) |
| Median | 5.70 | −1.20 | −29.27 | 4.00 | −0.80 | −15.47 |
| Min, Max | 0.2, 64.6 | −87.7, 15.5 | −92.8, 187.3 | 0.2, 81.0 | −37.9, 29.1 | −99.0, 740.0 |
| p-value | | | | | 0.1504 | 0.2078 |

Note:
p-value derived from a Wilcoxon Rank Sum Test comparing differences in distributions of changes (% Changes) between treatment group and placebo.

TABLE 7
Summary of CRP for Prednisolone Vs. 10 mg Paroxetine
ITT Population (LOCF)

| | 3 mg Prednisolone + Placebo | | | 3 mg Prednisolone + 10 mg Paroxetine | | |
|---|---|---|---|---|---|---|
| | (N = 69) | | | (N = 71) | | |
| | | Change from | % Change from | | Change from | % Change from |
| Visit | Value | baseline | baseline | Value | baseline | baseline |
| Baseline | | | | | | |
| N | 69 | | | 71 | | |
| Mean | 18.83 | | | 10.92 | | |
| (Std Dev) | (23.749) | | | (13.321) | | |
| Median | 8.90 | | | 6.50 | | |
| Min, Max | 0.3, 135.3 | | | 0.2, 58.7 | | |
| Day 14 | | | | | | |
| N | 69 | 69 | 69 | 71 | 71 | 71 |
| Mean | 11.42 | −7.40 | 7.11 | 7.74 | −3.18 | −15.70 |
| (Std Dev) | (17.413) | (20.149) | (215.970) | (10.712) | (7.126) | (71.753) |
| Median | 6.20 | −1.70 | −32.26 | 4.40 | −1.00 | −34.88 |
| Min, Max | 0.3, 107.3 | −83.2, 77.9 | −95.8, 1700.0 | 0.0, 49.7 | −27.0, 19.2 | −100.0, 316.0 |
| p-value | | | | | 0.4456 | 0.7128 |
| Day 42 | | | | | | |
| N | 69 | 69 | 69 | 71 | 71 | 71 |
| Mean | 10.22 | −8.61 | −17.67 | 9.54 | −1.38 | 27.51 |
| (Std Dev) | (12.443) | (18.009) | (58.589) | (14.300) | (10.398) | (288.446) |
| Median | 5.40 | −1.00 | −23.53 | 4.20 | −0.80 | −28.80 |
| Min, Max | 0.3, 70.6 | −81.4, 21.5 | −92.5, 187.3 | 0.2, 72.0 | −35.8, 55.3 | −86.3, 2340.0 |
| p-value | | | | | 0.1384 | 0.3673 |
| Day 70 | | | | | | |
| N | 69 | 69 | 69 | 71 | 71 | 71 |
| Mean | 9.88 | −8.95 | −13.66 | 9.82 | −1.09 | 16.45 |
| (Std Dev) | (11.956) | (18.996) | (64.905) | (14.480) | (7.437) | (120.350) |
| Median | 5.70 | −1.20 | −29.27 | 3.90 | −0.60 | −15.38 |
| Min, Max | 0.2, 64.6 | −87.7, 15.5 | −92.8, 187.3 | 0.2, 81.0 | −22.4, 29.1 | −87.5, 740.0 |
| p-value | | | | | 0.0753 | 0.1072 |

Note:
p-value derived from a Wilcoxon Rank Sum Test comparing differences in distributions of changes (% Changes) between treatment group and placebo.

TABLE 8
Summary of CRP for Prednisolone Vs. 20 mg Paroxetine
ITT Population (LOCF)

| | 3 mg Prednisolone + Placebo | | | 3 mg Prednisolone + 20 mg Paroxetine | | |
|---|---|---|---|---|---|---|
| | (N = 69) | | | (N = 69) | | |
| | | Change from | % Change from | | Change from | % Change from |
| Visit | Value | baseline | baseline | Value | baseline | baseline |
| Baseline | | | | | | |
| N | 69 | | | 69 | | |
| Mean | 18.83 | | | 13.90 | | |
| (Std Dev) | (23.749) | | | (17.938) | | |
| Median | 8.90 | | | 6.80 | | |
| Min, Max | 0.3, 135.3 | | | 0.7, 91.6 | | |
| Day 14 | | | | | | |
| N | 69 | 69 | 69 | 69 | 69 | 69 |
| Mean | 11.42 | −7.40 | 7.11 | 9.00 | −4.90 | −18.26 |
| (Std Dev) | (17.413) | (20.149) | (215.970) | (12.540) | (11.850) | (88.400) |
| Median | 6.20 | −1.70 | −32.26 | 4.60 | −1.70 | −41.28 |
| Min, Max | 0.3, 107.3 | −83.2, 77.9 | −95.8, 1700.0 | 0.3, 70.0 | −63.8, 29.0 | −85.2, 522.2 |
| p-value | | | | | 0.9441 | 0.2967 |
| Day 42 | | | | | | |
| N | 69 | 69 | 69 | 69 | 69 | 69 |
| Mean | 10.22 | −8.61 | −17.67 | 10.26 | −3.64 | −20.86 |
| (Std Dev) | (12.443) | (18.009) | (58.589) | (14.320) | (8.566) | (56.199) |
| Median | 5.40 | −1.00 | −23.53 | 4.50 | −1.20 | −27.42 |
| Min, Max | 0.3, 70.6 | −81.4, 21.5 | −92.5, 187.3 | 0.1, 64.0 | −31.9, 17.5 | −97.6, 202.1 |
| p-value | | | | | 0.6771 | 0.7840 |
| Day 70 | | | | | | |
| N | 69 | 69 | 69 | 69 | 69 | 69 |
| Mean | 9.88 | −8.95 | −13.66 | 11.44 | −2.46 | −4.72 |
| (Std Dev) | (11.956) | (18.996) | (64.905) | (14.834) | (9.372) | (88.625) |
| Median | 5.70 | −1.20 | −29.27 | 5.30 | −1.10 | −16.67 |
| Min, Max | 0.2, 64.6 | −87.7, 15.5 | −92.8, 187.3 | 0.2, 64.0 | −37.9, 25.3 | −99.0, 482.5 |
| p-value | | | | | 0.4888 | 0.5808 |

Note:
p-value derived from a Wilcoxon Rank Sum Test comparing differences in distributions of changes (% Changes) between treatment group and placebo.

TABLE 9
Summary of Patient Pain Assessment (VAS) Scores for Prednisolone Vs.
Combined Paroxetine Groups
ITT Population (LOCF)

| | 3 mg Prednisolone + Placebo | | | 3 mg Prednisolone + 10-20 mg Paroxetine | | |
|---|---|---|---|---|---|---|
| | (N = 69) | | | (N = 140) | | |
| | | Change from | % Change from | | Change from | % Change from |
| Visit | Value | baseline | baseline | Value | baseline | baseline |
| Baseline | | | | | | |
| N | 69 | | | 140 | | |
| Mean | 53.31 | | | 52.31 | | |
| (Std Dev) | (22.493) | | | (22.861) | | |
| Median | 52.00 | | | 49.00 | | |
| Min, Max | 7.0, 97.0 | | | 7.0, 98.0 | | |
| Day 14 | | | | | | |
| N | 69 | 69 | 69 | 140 | 140 | 140 |
| Mean | 45.58 | −7.73 | −13.65 | 41.85 | −10.46 | −14.56 |
| (Std Dev) | (25.283) | (19.337) | (38.137) | (23.243) | (21.686) | (45.212) |
| Median | 47.00 | −5.00 | −11.11 | 40.50 | −7.00 | −13.21 |
| Min, Max | 2.0, 97.0 | −81.0, 35.0 | −94.7, 106.7 | 1.0, 93.0 | −79.0, 40.0 | −94.4, 205.6 |
| p-value | | | | | 0.3842 | 0.5690 |
| Day 42 | | | | | | |
| N | 69 | 69 | 69 | 140 | 140 | 140 |
| Mean | 42.74 | −10.57 | −19.23 | 33.37 | −18.94 | −33.53 |
| (Std Dev) | (26.143) | (21.630) | (47.955) | (25.041) | (22.540) | (47.151) |
| Median | 37.00 | −9.00 | −23.44 | 27.00 | −18.00 | −37.26 |
| Min, Max | 2.0, 97.0 | −64.0, 56.0 | −93.5, 186.7 | 0.0, 97.0 | −85.0, 36.0 | −100.0, 163.2 |
| p-value | | | | | 0.0206 | 0.0200 |
| Day 70 | | | | | | |
| N | 69 | 69 | 69 | 140 | 140 | 140 |
| Mean | 41.04 | −12.28 | −17.84 | 37.10 | −15.21 | −22.72 |
| (Std Dev) | (25.466) | (23.864) | (57.121) | (27.863) | (27.066) | (68.982) |
| Median | 39.00 | −10.00 | −25.64 | 31.00 | −13.00 | −29.11 |
| Min, Max | 2.0, 97.0 | −69.0, 49.0 | −90.5, 233.3 | 0.0, 97.0 | −91.0, 70.0 | −100.0, 466.7 |
| p-value | | | | | 0.4716 | 0.3293 |

Note:
p-value derived from a Wilcoxon Rank Sum Test comparing differences in distributions of changes (% Changes) between treatment group and placebo.

TABLE 10
Summary of Patient Pain Assessment (VAS) Scores for Prednisolone Vs. 10 mg Paroxetine
ITT Population (LOCF)

| | 3 mg Prednisolone + Placebo | | | 3 mg Prednisolone + 10 mg Paroxetine | | |
|---|---|---|---|---|---|---|
| | (N = 69) | | | (N = 71) | | |
| | | Change from | % Change from | | Change from | % Change from |
| Visit | Value | baseline | baseline | Value | baseline | baseline |
| Baseline | | | | | | |
| N | 69 | | | 71 | | |
| Mean | 53.31 | | | 51.05 | | |
| (Std Dev) | (22.493) | | | (20.405) | | |
| Median | 52.00 | | | 50.00 | | |
| Min, Max | 7.0, 97.0 | | | 9.0, 93.0 | | |
| Day 14 | | | | | | |
| N | 69 | 69 | 69 | 71 | 71 | 71 |
| Mean | 45.58 | −7.73 | −13.65 | 45.15 | −5.89 | −4.34 |
| (Std Dev) | (25.283) | (19.337) | (38.137) | (21.788) | (19.259) | (48.497) |
| Median | 47.00 | −5.00 | −11.11 | 46.00 | −3.00 | −4.35 |
| Min, Max | 2.0, 97.0 | −81.0, 35.0 | −94.7, 106.7 | 3.0, 93.0 | −42.0, 40.0 | −93.2, 205.6 |
| p-value | | | | | 0.6820 | 0.4165 |
| Day 42 | | | | | | |
| N | 69 | 69 | 69 | 71 | 71 | 71 |
| Mean | 42.74 | −10.57 | −19.23 | 35.84 | −15.21 | −27.16 |
| (Std Dev) | (26.143) | (21.630) | (47.955) | (24.433) | (20.510) | (47.385) |
| Median | 37.00 | −9.00 | −23.44 | 29.00 | −14.00 | −28.57 |
| Min, Max | 2.0, 97.0 | −64.0, 56.0 | −93.5, 186.7 | 0.0, 97.0 | −63.0, 36.0 | −100.0, 163.2 |
| p-value | | | | | 0.2255 | 0.3060 |
| Day 70 | | | | | | |
| N | 69 | 69 | 69 | 71 | 71 | 71 |
| Mean | 41.04 | −12.28 | −17.84 | 39.35 | −11.70 | −20.92 |
| (Std Dev) | (25.466) | (23.864) | (57.121) | (28.086) | (24.235) | (51.283) |
| Median | 39.00 | −10.00 | −25.64 | 32.00 | −13.00 | −24.62 |
| Min, Max | 2.0, 97.0 | −69.0, 49.0 | −90.5, 233.3 | 0.0, 97.0 | −65.0, 50.0 | −100.0, 119.0 |
| p-value | | | | | 0.9502 | 0.9453 |

Note:
p-value derived from a Wilcoxon Rank Sum Test comparing differences in distributions of changes (% Changes) between treatment group and placebo.

TABLE 11
Summary of Patient Pain Assessment (VAS) Scores for Prednisolone Vs. 20 mg Paroxetine
ITT Population (LOCF)

| | 3 mg Prednisolone + Placebo | | | 3 mg Prednisolone + 20 mg Paroxetine | | |
|---|---|---|---|---|---|---|
| | (N = 69) | | | (N = 69) | | |
| | | Change from | % Change from | | Change from | % Change from |
| Visit | Value | baseline | baseline | Value | baseline | baseline |
| Baseline | | | | | | |
| N | 69 | | | 69 | | |
| Mean | 53.31 | | | 53.61 | | |
| (Std Dev) | (22.493) | | | (25.226) | | |
| Median | 52.00 | | | 49.00 | | |
| Min, Max | 7.0, 97.0 | | | 7.0, 98.0 | | |
| Day 14 | | | | | | |
| N | 69 | 69 | 69 | 69 | 69 | 69 |
| Mean | 45.58 | −7.73 | −13.65 | 38.46 | −15.15 | −25.07 |
| (Std Dev) | (25.283) | (19.337) | (38.137) | (24.342) | (23.139) | (39.195) |
| Median | 47.00 | −5.00 | −11.11 | 36.00 | −10.00 | −24.62 |
| Min, Max | 2.0, 97.0 | −81.0, 35.0 | −94.7, 106.7 | 1.0, 88.0 | −79.0, 34.0 | −94.4, 63.0 |
| p-value | | | | | 0.0534 | 0.0689 |
| Day 42 | | | | | | |
| N | 69 | 69 | 69 | 69 | 69 | 69 |
| Mean | 42.74 | −10.57 | −19.23 | 30.83 | −22.78 | −40.08 |
| (Std Dev) | (26.143) | (21.630) | (47.955) | (25.580) | (24.001) | (46.338) |
| Median | 37.00 | −9.00 | −23.44 | 23.00 | −20.00 | −49.38 |
| Min, Max | 2.0, 97.0 | −64.0, 56.0 | −93.5, 186.7 | 0.0, 87.0 | −85.0, 26.0 | −100.0, 118.2 |
| p-value | | | | | 0.0052 | 0.0027 |
| Day 70 | | | | | | |
| N | 69 | 69 | 69 | 69 | 69 | 69 |
| Mean | 41.04 | −12.28 | −17.84 | 34.78 | −18.83 | −24.58 |
| (Std Dev) | (25.466) | (23.864) | (57.121) | (27.643) | (29.440) | (83.742) |
| Median | 39.00 | −10.00 | −25.64 | 29.50 | −13.00 | −40.38 |
| Min, Max | 2.0, 97.0 | −69.0, 49.0 | −90.5, 233.3 | 0.0, 92.0 | −91.0, 70.0 | −100.0, 466.7 |
| p-value | | | | | 0.1861 | 0.1034 |

Note:
p-value derived from a Wilcoxon Rank Sum Test comparing differences in distributions of changes (% Changes) between treatment group and placebo.

### OTHER EMBODIMENTS

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method for reducing pain in a patient in need thereof, said method comprising administering to said patient (i) a corticosteroid; and (ii) an SSRI, wherein said corticosteroid and said SSRI are administered in amounts and for a duration that together are sufficient to reduce pain in said patient.

2. The method of claim 1, wherein said pain is inflammatory pain, neuropathic pain, or nociceptive pain.

3. The method of claim 1, wherein said pain is dysfunctional pain caused by fibromyalgia, tension type headache, irritable bowel disorders, or migraine.

4. The method of claim 1, wherein said corticosteroid is algestone, 6-alpha-fluoroprednisolone, 6-alpha-methylprednisolone, 6-alpha-methylprednisolone 21-acetate, 6-alpha-methylprednisolone 21-hemisuccinate sodium salt, 6-alpha, 9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-17-valerate, budesonide, clobetasol, clobetasol propionate, clobetasone, clocortolone, clocortolone pivalate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, flupredidene, fluprednisolone, flurandrenolide, formocortal, halcinonide, halometasone, halopredone, hyrcanoside, hydrocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methylprednisolone, methylprednisolone acetate, methylprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-21-acetate, prednisolone-21 (beta-D-glucuronide), prednisone, prednylidene, procinonide, tralonide, triamcinolone, triamcinolone acetonide, triamcinolone acetonide 21-palmitate, triamcinolone diacetate, triamcinolone hexacetonide, and wortmannin.

5. The method of claim 1, wherein said SSRI is cericlamine, citalopram, clovoxamine, cyanodothiepin, dapoxetine, duloxetine, escitalopram, femoxetine, 4-(2-fluorophenyl)-6-methyl-2-piperazinothieno[2,3-d]pyrimidine, fluoxetine, fluvoxamine, ifoxetine, indalpine, indeloxazine, litoxetine, milnacipran, paroxetine, sertraline, venlafaxine, viqualine, or zimeldine.

6. The method of claim 1, wherein said corticosteroid and said SSRI are formulated in a single composition.

7. The method of claim 1, wherein said corticosteroid is in a low dosage.

8. The method of claim 1, wherein said composition is formulated for topical, systemic, or oral administration.

9. The method of claim 1, wherein said corticosteroid is prednisolone and said SSRI is paroxetine.

10. The method of claim 1, wherein 5 to 50 mg of paroxetine are administered to said patient daily.

11. The method of claim 1, wherein said prednisolone and paroxetine are formulated in separate dosage forms.

12. The method of claim 1, further comprising administering to said patient a third agent selected from the group consisting of antibiotics, disease-modifying anti-rheumatic drugs (DMARDs), non-steroidal anti-inflammatory drugs (NSAIDs), anti-convulsants, muscle relaxants, analgesics, cannibinoids, or sedatives.

13. A method for treating pruritus in a patient in need thereof, said method comprising administering to said patient (i) a corticosteroid; and (ii) an SSRI, wherein said corticosteroid and said SSRI are administered in amounts and for a duration that together are sufficient to treat said patient.

14. A kit comprising:
(i) a corticosteroid;
(ii) an SSRI; and
(iii) instructions for administering said corticosteroid and said SSRI to a patient for the treatment of pain.

15. A kit comprising:
(i) a corticosteroid; and
(ii) instructions for administering said corticosteroid with an SSRI to a patient for the treatment of pain.

16. A kit comprising:
(i) an SSRI; and
(ii) instructions for administering said SSRI with a corticosteroid to a patient for the treatment of pain.

17. A kit comprising:
(i) a composition comprising a corticosteroid and an SSRI; and
(ii) instructions for administering said composition to a patient for the treatment of pain.
